(19) United States
(12) Patent Application Publication
(10) Pub. No.: US 2017/0065554 A1
(43) Pub. Date: Mar. 9, 2017

(54) ACTIVATED SOY POD FIBER

(71) Applicant: MicroBlome Therapeutics LLC, Broomfield, CO (US)

(72) Inventors: Mark L. HEIMAN, Indianapolis, IN (US); Stephen M. BOUE, New Orleans, LA (US)

(21) Appl. No.: 15/120,381

(22) PCT Filed: Feb. 20, 2015
(86) PCT No.: PCT/US15/16968

§ 371 Date: Aug. 19, 2016

Related U.S. Application Data
(60) Provisional application No. 61/942,415, filed on Feb. 20, 2014.

(51) Int. Cl. A61K 31/352; A23L 33/00; A61K 9/14; A23L 33/21; A61K 36/48; A61K 9/00

(52) U.S. Cl. CPC A61K 31/352; A61K 36/48; A61K 9/0053; A61K 9/14; A23L 33/21; A23L 33/30; A23V 2002/00

(57) ABSTRACT

The present disclosure encompasses, among other things, compositions and methods of enhancing glyceollin content of soybean tissue as well as methods of large-scale production of soybean tissues comprising elevated glyceollin content. The present disclosure further encompasses administration of food products comprising glyceollins in the treatment or prevention (i.e., delay of onset) of overweight, obesity, prediabetes, diabetes, IBD, and gastrointestinal dysbiosis.

### CROSS REFERENCE TO RELATED APPLICATIONS

This Application claims the benefit of priority to U.S. Provisional Application No. 61/942,415, filed Feb. 20, 2014, the entire contents of which are hereby incorporated by reference in their entirety for all purposes.

### BACKGROUND

One of the benefits of modern technology is that human longevity and health have improved, but modern society promotes more sedentary occupations. Before industrialization and the transition from farm to metropolis, the human body did the work.

Modern agricultural systems are developed with two related goals in mind: to obtain the highest yields possible and to get the highest economic profit possible. Benefits of food processing include preservation, efficient marketing and distribution, and increasing food consistency. In addition, it increases yearly availability of many foods, enables transportation of delicate perishable foods across long distances and makes many kinds of foods safe to eat by de-activating spoilage and pathogenic micro-organisms. An unfortunate consequence of modern food processing is that more calories are packed into a gram of food than ever before.

As a consequence of modernization, people of modern societies live longer, consume more calories in a day and are more sedentary. There is a great risk that loss of metabolic fitness accompanies modernization. Each cell of the body has an important function and it consumes energy to perform that function. The energy is supplied by metabolism of food and often measured by heat units or calories. Energy balance is a point when the total energy supplied by diet matches the total energy demand of all cells. To prepare for periods of negative energy balance or when the total energy spent is greater than total energy consumed, energy is converted to fat and it is stored in fat cells or adipocytes. Storage occurs during periods of positive energy balance or when total calories consumed and absorbed are greater than total calories utilized during that period. A metabolically fit individual is one who consumes sufficient calories to meet the energy demand and deposit excess calories as fat in adipocytes. However, a consequence of modernization is often a loss of metabolic fitness. There is such an abundance of calories consumed that the adipocytes become overloaded and fat synthesized for storage is hoarded in other tissues.

While it is perfectly healthy to store fat in adipocytes, it is unhealthy to store fat in any other organ like liver, arteries, pancreas, muscle, bone, brain, etc. Modernization has resulted in discovery of new methods to measure the level of fat supply and to locate where fat is being accumulated. An inexpensive method is to measure height and body weight to calculate a body mass index or BMI. It appears one is metabolically fit when BMI is about 25. But a loss of fitness is observed when BMI is greater than 25. Obesity is defined when BMI is 30 or greater. It is the consequence of fat stored in tissues other than adipocytes that is unhealthy and there are numerous studies using BMI as an index to monitor and correlate many pathological findings, such as diabetes and cardiovascular diseases.

Diabetes affects nearly 25.8 million people or 8.3% of the U.S. population and is projected to rise significantly over the next decade. In most cases, the diabetes results from excess fat stored in tissues that utilize insulin to supply them with energy (glucose) needed for their specialized function. Those tissues become increasing less sensitive to insulin as they accumulate fat and glucose remains in the blood. This is termed type 2 diabetes (T2D). The global incidence of T2D is at a pandemic rate as more societies become modernized. Diabetes is the seventh leading cause of death in the U.S. In addition to these staggering mortality data, diabetes results in devastating morbidities that result in high healthcare costs. Indeed, after adjusting for population, age, and sex differences, average medical expenditures among people with diabetes were 2.3-times higher than what expenditures would be in the absence of diabetes. Notably, a 2007 estimate suggests that the total (direct and indirect) estimated cost for diabetes was $174 billion in that year alone.

In order to better educate non-diabetic patients about their potential for progressing toward a clinical case of diabetes, the Centers for Disease Control (CDC) and the American Diabetes Association (ADA) coined the term “prediabetes”. In this way, medical practitioners can identify patients at higher risk for developing diabetes. Those patients typically have a BMI between 25 and 35 and T2D is closely associated with BMIs between 30 and 45. The CDC estimates that there are 79 million Americans aged 20 years or older with prediabetes. Without intervention, about 11% are expected progress to type 2 diabetes (T2D) in just 3 years. Prediabetes is defined by the ADA as fasting blood glucose levels between 100 mg/dl and 125 mg/dl, or blood glucose level between 140 mg/dl and 125 mg/dl 2 h after an oral glucose tolerance test (OGTT) and a hemoglobin A1c level between 5.7% and 6.4%. Data exist to support that targeted treatment regimens for prediabetics can significantly reduce the risk of progressing to T2D. For example, the Diabetes Prevention Program (DPP) demonstrated that prediabetics who received intensive counseling on diet, exercise, and behavior modification were able to reduce their risk of developing diabetes by 58 percent and those who took metformin reduced the risk of developing diabetes by 31 percent [3]. Moreover, if reversion to normal glucose regulation occurred only transiently, there was a significantly reduced risk of progressing to diabetes. Thus, there is a need for additional pre diabetes interventions that are inexpensive, safe, and efficacious.

In less common cases, diabetes is a consequence of the natural immune system attacking the pancreatic cells that produce and secrete insulin—the pancreatic beta (β) cells. This type of diabetes is called Type I Diabetes (T1D) or Type 1 juvenile diabetes mellitus. It is characterized by the infiltration of activated T lymphocytes and monocytes into the islets of Langerhans of the pancreas, resulting in inflammation and progressive destruction of the insulin-producing β cells. The typical diet for an American, especially an American child, is deficient in both edible plant fiber and plant polyphenols. Thus, the GI microbiota that utilize those substrates will be at a disadvantage and therefore, less abundant than the other inhabitants of that biome. Dietary intervention by replacing those essential food constituents may prove to be beneficial in the prevention and (or) management of type I diabetes by modulating the GI microbiome.

Another consequence of the modern diet is increased prevalence of Inflammatory Bowel Disease (IBD). The incidence and prevalence of IBD are increasing with time and in different regions around the world, indicating its emergence as a global disease. (Molodecky et al., Gastroenterol 142:46-54, 2012). IBD is a collective term that describes conditions with chronic or recurring immune response and inflammation of the gastrointestinal (GI) tract. The two most common inflammatory bowel diseases are ulcerative colitis (UC) and Crohn's disease (CD). Both are marked by an abnormal response of the GI immune system. Normally, immune cells protect the body from infection. In people with IBD, however, this immune system mistakes food, bacteria, and other materials in the intestine for pathogens and an inflammatory response is launched into the lining of the intestines creating chronic inflammation. When this happens, the patient experiences the symptoms of IBD. The highest reported prevalence values for IBD were in Europe (UC, 505 per 100,000 persons; CD, 322 per 100,000 persons) and North America (UC, 249 per 100,000 persons; CD, 319 per 100,000 persons).

At the end of 2007, the US National Institutes of Health (NIH) launched the Human Microbiome Project (HMP) and, in early 2008, the European Commission and China initiated the Metagenomics project of the Human Intestinal Tract (MetaHIT). These large efforts apply advanced sequencing and bioinformatic tools to characterize the microbes living in and on our bodies. An estimated 100 trillion microorganisms reside in the large intestine where they play a role in metabolizing food and converting it to energy for cellular work or to be banked in reserve. Understanding how the activities of these microbial populations impact human metabolism may offer approaches to develop interventions to prevent metabolic unfitness and to treat obesity, diabetes and IBD.

For example, Chinese T2D patients have recently been characterized with a moderate degree gastrointestinal (GI) bacterial dysbiosis or an abnormal population of microbiota. Analysis reveals that the GI microbiota of those T2Ds have decreased ability to synthesize short chain fatty acids (SCFAs), an increased ability to produce hydrogen sulfide, an increased ability to produce methane, and decreased defense against oxidative stress. Increased production of hydrogen sulfide is also associated with IBD. A metabolically unfit population would benefit from supplementing their diets with poorly absorbed antioxidants. One class of antioxidant polyphenolics called isoflavones is produced by soy plants and is shown to promote health in humans. For example, the isoflavones genistein, daidzein, and glycitein are in particularly high levels in traditional soy-based foods. Consumption of a diet rich in soy products may prevent T2D and IBD. Other isoflavones are induced by the plant's defense mechanisms. Those compounds are termed phytoalexins. Three very similar phytoalexins called glyceollin I, glyceollin II, and glyceolin III, are produced by soy when the plant is exposed to soil microorganisms, ultraviolet (UV) light or heavy metals and are very potent antioxidants (19). Supplementation of the diet for a prediabetic or a patient with IBD with the glyceollins will deliver an antioxidant to the GI biome. Without wishing to be bound by theory, it is believed herein that changing the redox potential of the GI biome that selects for families of microbiota will benefit the prediabetic and the IBD patient.

### SUMMARY

The present invention provides, among other things, compositions comprising isolated plant tissue having glyceollin

In some embodiments, a composition comprising isolated soy pod tissue containing one or more glyceollins is provided. In some embodiments, the combined total concentration of one or more glyceollins in the soy pod tissue is at least 0.01, 0.05, 0.1, 0.5, 1.0, or 5.0 mg per gram.

In some embodiments, isolated soy pod containing both soluble and insoluble dietary fiber is provided.

In some embodiments, isolated soy pod formulated for oral delivery is provided.

In some embodiments, the invention provides a food product comprising dietary fiber from soy pod tissue. In some embodiments, the food product comprises one or more glyceollins. In some embodiments, the food product comprises glyceollins in a total amount of at least 2.5, 5.0, 7.5, 10.0, 20.0, 40.0 or 80.0 mg.

In some embodiments, the invention provides a powder comprising one or more glyceollins. In some embodiments, the powder is made from soy pod tissue. In some embodiments, the powder comprises one or more glyceollins at a combined total concentration of at least 0.01, 0.05, 0.1, 0.5, 1.0, or 5.0 mg glyceollins per gram of powder.

The invention further provides methods for treating a subject suffering from or

susceptible to overweight or obesity. In some embodiments, the methods comprise orally administering to the subject a composition or food product as described herein.

The invention further provides methods for treating subject suffering from or susceptible to diabetes, or prediabetes. In some embodiments, the methods comprise orally administering to the subject a composition or food product as described herein.

The invention further provides methods for treating subject suffering from or susceptible to IBD. In some embodiments, the methods comprise orally administering to the subject a composition or food product as described herein.

The invention further provides methods for modifying the gastrointestinal microbiome of a subject, wherein the gastrointestinal microbiome of the subject includes a first population of bacteria that process fat and protein, and a second population of bacteria that ferment carbohydrate and produce increases in small chain fatty acids. In some embodiments, the method comprises administering to the subject a composition comprising an effective amount of one or more glyceollins to shift the relative abundance of the first population of bacteria and the second population of bacteria in the gastrointestinal tract.

In some embodiments, the first population comprises Ruminococcaceae and the second population comprises Blautia hydrogenotrophica.

In some embodiments, methods are provided for modifying the level of Blautia in the microbiota taxa of a subject. In some embodiments, a subject is identified as having Blautia level below 2, 3, 4, or 5% abundance and in need of treatment with an effective amount of one or more glyceollins to increase Blautia levels to at least 10%, 15%, 20%, 25%, or 30% abundance. In some embodiments, a subject identified as in need of treatment is administered a composition comprising one or more glyceollins to increase Blautia levels. In some embodiments, methods for treating gastrointestinal dysbiosis are provided comprising the step of orally administering to the subject an effective amount of a composition comprising one or more glyceollins.

The invention further provides methods of manufacturing a powder comprising soypod dietary fiber and one or more glyceollins. In some embodiments the method comprises the steps of obtaining soy pod tissue, slicing the soy pod tissue, drying the soy pod tissue, and milling the soy pod tissue into a powder. In some embodiments, the method comprises adding one or more glyceollins to the soy pod tissue. In some embodiments, the method comprises exposing the soy pod tissue to ultraviolet radiation, heavy metals or fungi infection.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The following figures are presented for the purpose of illustration only, and are not intended to be limiting.

FIG. 1 shows exemplary results illustrating plasma levels of glyceollins in ZDSD/Pco rats after administration of glyceollins (30 and 90 mg/kg, p.o.). Values represent the mean±SEM from 3 different rats at each time point and dose.

FIG. 2 shows exemplary results illustrating blood glucose levels of prediabetic ZDSD/Pco rats after administration of glucose (2 g/kg, p.o., at time 0). Glyceollins were administered (30 and 90 mg/kg, p.o.) 1 h prior to the start of the oral glucose tolerance test. Each symbol represents the mean±SEM of the blood glucose value for 8 rats. At 60 min, the blood glucose levels for the glyceollin treated animals were significantly lower than those for the vehicle-treated rats, and the areas under the curves for the glyceollin groups were significantly less than that integrated for the vehicle-treated rats.

FIG. 3 shows exemplary results illustrating insulin-mediated glucose uptake by 3T3-L1 adipocytes. Cells were exposed to insulin for 30 min at 37° C. followed by 10 min of incubation with [³H]-2Deoxy-glucose. The effective concentration for 50% increase in glucose uptake (EC₅₀) was 1.92 nM when computed by the 4-parameter logistic equation using SigmaPlot.

These data are the average of 6 experiments that were normalized by calculating the percent cpm glucose uptake compared to basal cpm glucose uptake. The symbols represent mean±SEM and the line represents the best fit to the data using the 4-parameter logistic equation.

FIG. 4 shows exemplary results illustrating insulin, glyceollins, and insulin combined with glyceollins stimulated glucose uptake by 3T3-L1 adipocytes. Adipocytes were exposed to inulin, glyceollins, or both for 3 h. These data are the average of 3 experiments that were normalized by calculating the percent cpm glucose uptake compared to basal cpm glucose uptake. The symbols represent mean±SEM. All means for insulin-stimulated glucose uptake with different letters are significantly (p<0.05) different.

FIG. 5 shows exemplary results illustrating glyceollin-mediated glucose uptake by 3T3-L1 adipocytes. Cells were exposed to glyceollin for 45 min at 37° C. followed by 10 min of incubation with [³H]-2-deoxy-glucose. The EC₅₀ was 2.40±0.43 μM and a maximal uptake of 2.04±0.24-fold (computed by the 4-parameter logistic equation). These data are the average of 3 experiments that were normalized by calculating the percent cpm glucose uptake compared to basal cpm glucose uptake. The symbols represent mean±SEM and the line represents the best fit to the data using the 4-parameter logistic equation.

FIG. 6 shows exemplary results illustrating glyceollins stimulate the expression of glucose transporter genes GLUT1 and GLUT4 in 3T3-L1 adipocytes. mRNA levels of both genes were measured by real time PCR and are shown relative to mRNA level of RPL32. The cells were exposed to glyceollin for 3 h, mRNA was isolated from the cells, cDNA was synthesized, and gene expression was quantitated by real time PCR. Symbols represent mean±SEM.

FIG. 7 shows exemplary results illustrating daily administration of a glyceollin blend (90 mg/kg, p.o.) decreases fat mass of prediabetic rats by 11 days. Fat mass was measured by quantitative NMR.

FIG. 8 shows exemplary results illustrating daily administration of the glyceollin blend (90 mg/kg, p.o.) tends to increase plasma GLP-1 of prediabeticrats by 11 days. GLP-I was measured in trunk blood by ELISA at the end of the study.

FIG. 9 shows exemplary results illustrating daily administration of the glyceollin blend (90 mg/kg, p.o.) increases plasma insulin of prediabetic rats by 11 days. Plasma insulin was measured in trunk blood by ELISA at the end of the study and plasma glucose was measured by glucometer.

FIG. 10 shows exemplary results illustrating an HPLC chromatogram revealing compounds present in soy pod fiber after 0 to 72 incubation following slicing into 1 mm cross sections. Peaks 13, 14 and 15, which elute at about 32 min are Glyceollin III, Glyceollin II and Glyceollin I, respectively. The soy pods were processed at laboratory scale under analytical conditions.

FIG. 11 shows exemplary results illustrating laboratory scale production of activated soy pod (Iowa edamame variety (IA)) fiber after 72 hours of incubation following slicing into 1 mm cross sections and exposing to uv-B radiation for 2 min.

FIG. 12. HPLC chromatogram of milled unactivated soy pod fiber (Red) and activated soy pod fiber (Blue). Peaks from 35-36 min represent unidentified isoflavones that are also generated by the activation process.

FIG. 13. Phenotypes of mice consuming the obesogenic diet (ObD) that was supplemented with activated soy pod fiber (ObD-ASPF) or unactivated soy pod fiber (ObD-USPF). Food intake (A) was increased by week 4 in mice fed either ObD-ASPF or ObD-USPF. Body weight gain (B) tended to be less in the ObD-ASPF and ObD-USPF groups. Body weight gained was a consequence of both fat (C) and lean (D) mass accretion. Symbols are the mean±SEM. * p=0.012 vs. ObD, ** p=0.037 vs. ObD. Bars are the mean±SEM. Levels not connected by the same letter are statistically different (p<0.05). N=10 for each group.

FIG. 14. Loss of energy in feces. Fecal output (A) was evaluated weekly and both fecal caloric density (B) and fecal triglyceride (TG) content (C) was evaluated at baseline and after 4 weeks (final) when mice consumed the obesogenic diet (ObD), which was supplemented with activated soy pod fiber (ObD-ASPF) or unactivated soy pod fiber (ObD-USPF). Symbols and bars are the mean±SEM. Levels not connected by the same letter are statistical different (p<0.05). n=10 for each group.

FIG. 15. Fecal total bile acids content was decreased in the ObD-supplemented diets. Fecal bile acid content was measured at baseline and after 4 weeks (final) when mice consumed the obesogenic diet (ObD), which was supplemented with activated soy pod fiber (ObD-ASPF) or unactivated soy pod fiber (ObD-USPF). Bars are the mean±SEM. Levels not connected by the same letter are statistical different (p<0.05). n=10 for each group.

FIG. 16. Gut microbiota species in genera Flavonifactor (A), Barnesiella (B). Bacteroides (C). Oscillibactor (D), and Alistipes (E) significantly increase from baseline to 4 weeks (final) of feeding the obesogenic diet (ObD), supplemented with activated soy pod fiber (ObD-ASPF) or supplemented with unactivated soy pod fiber (ObD-USPF). Bars are the mean±SEM. Levels not connected by the same letter are statistical different (p<0.05). Abundances were the mean number of reads from fecal samples of all 10 mice in each group.

FIG. 17. Increased fermentation from baseline to 4 weeks (final) of feeding the ObD-ASPF) or ObD-USPF. Evidence includes decreased fecal pH (A) with increased fecal short chain fatty acid content for acetate (B), butyrate (C) and propionate (D). Bars are the mean±SEM. Levels not connected by the same letter are statistical different (p<0.05). n=10 for each of the 3 groups.

FIG. 18. Gut microbiota species in genera Parabacterodies (A), Ruminococcus (B), Hydrogenoanaerobacterium (C), and Lactococcus (D) significantly decrease in abundance from baseline to 4 weeks (final) of feeding ObD-ASPF or ObD-USPF. Bars are the mean±SEM. Levels not connected by the same letter are statistical different (p<0.05). Abundances were the mean number of reads from fecal samples of all 10 mice in each group.

FIG. 19. Shifts in fecal lactate (A) and fecal glucose (B) content from baseline after 4 weeks (final) of feeding ObD-ASPF or ObD-USPF. Bars are the mean±SEM. Levels not connected by the same letter are statistical different (p<0.05). n=10 for each group.

FIG. 20. Gut microbiota genera Akkermanisia and Mucispirillum of the Verrucomicrobia and Deferribacteres phyla, respectively, significantly shift from baseline after 4 weeks (final) of feeding ObD-ASPF or ObD-USPF. Bars are the mean±SEM. Levels not connected by the same letter are statistical different (p<0.05). Abundances were the mean number of reads from fecal samples of all 10 mice in each group.

FIG. 21. Increased secretory immunoglobulin A content in feces of mice fed ObD-USPF for 4 weeks (final). Bars are the mean±SEM. Levels not connected by the same letter are statistical different (p<0.05). n=10 for each of the 3 groups.

FIG. 22. Plasma proinflammatory levels after 4 weeks of feeding ObD, ObD-ASPF or ObD-USPF. Bars are the mean±SEM. *p=0.01, ** p=0.03. n=10 for each of the 3 groups.

FIG. 23. Hematoxylin and eosin stain of proximal colon from even numbered mice consuming ObD, ObD-ASPF and ObD-USPF for 30 days. A histologist, who was masked to treatments, identified similar views of a crypt from each mouse for comparison. Sections are magnified 400×.

FIG. 24. Table detailing composition of purified mouse diets. The regularly used obesogenic condensed milk fat diet (D12266B) was modified with Activated- or Unactivated-Soy Pod Fiber by Research Diets, Inc.

### DEFINITIONS

In order for the present invention to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Amelioration: As used herein, the term “amelioration” means the prevention, reduction or palliation of a state, or improvement of the state of a subject. Amelioration includes, but does not require, complete recovery or complete prevention of a disease condition.

Comparable: As used herein, the term “comparable” refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

Correlates: As used herein, the term “correlates”, refers to its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

Dysbiosis or Gastrointestinal dysbiosis: As used herein, the term “dysbiosis” (also called dysbacteriosis) as used herein refers to a condition when a microbial population occupying a habitat on or in the body during health is shifted to a population of microbiota identified in the same habitat in an unhealthy or diseased state. Dysbiosis is most prominent in the digestive tract (also called gastrointestinal dysbiosis) where it is associated with illnesses such as diabetes, obesity, irritable bowel syndrome, inflammatory bowel disease and gastric ulcers.

Food product: As used herein, the term “food product” refers to food or a food ingredient that is specially formulated and intended for the dietary management of a disease that has distinctive nutritional needs that cannot be met by normal diet alone.

Glyceollins: As used herein, the term “glyceollins” refers to the phytoalexins glyceollin I, glyceollin II, and glyceolin III, and similar compounds that are potent antioxidants produced in soy when the plant is cut, exposed to soil microorganisms, exposed to ultraviolet (UV) light or exposed to heavy metals. Phytoalexins are isoflavones that are induced by a plant's defense mechanisms.

Improve, increase, or reduce: As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of treatment described herein, or a measurement in a control individual (or multiple control individuals) in the absence of treatment) described herein. In some embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with overweight, obesity, prediabetes, diabetes, or gastrointestinal dysbiosis, who is about the same age and/or gender as the individual being treated (to ensure that the stages of the disease in the treated individual and the control individual(s) are comparable).

Microbiome or Gastrointestinal microbiome: As used herein, the term “microbiome” refers to the totality of microbes, their genetic elements (genomes), and environmental interactions in a particular environment (habitat or ecosystem). The term “gastrointestinal microbiome” refers to the microbiome of the gastrointestinal tract.

Prediabetes: As used herein, the term “prediabetes” refers to a condition in which individuals have fasting blood glucose or hemoglobin A1c levels higher than normal but not high enough to be diagnosed as diabetic. People with prediabetes have an increased risk of developing type 2 diabetes.

Inflammatory Bowel Disease (IBD): As used herein, the term “IBD” refers to conditions in which individuals have chronic or recurring immune response and inflammation of the gastrointestinal (GI) tract. The two most common inflammatory bowel diseases are ulcerative colitis (UC) and Crohn's disease (CD).

Providing: As used herein, the term “providing” refers to performing a manipulation that causes an entity of interest to be present at a level and/or with an activity higher than that observed under otherwise comparable conditions prior to or absent the manipulation. In some embodiments, providing consists of or comprises administering the entity itself (alone or as part of a composition); in some embodiments, providing consists of or comprises administering an agent that causes an increase in level and/or activity of the entity of interest.

Reference: A “reference” entity, system, amount, set of conditions, etc., is one against which a test entity, system, amount, set of conditions, etc. is compared as described herein. For example, in some embodiments, a “reference” individual is a control individual who is not suffering from or susceptible overweight, obesity, diabetes, or gastrointestinal dysbiosis; in some embodiments, a “reference” individual is a control individual afflicted with the same form of disease as an individual being treated, and optionally who is about the same age as the individual being treated (to ensure that the course of the disease or pre-diseased state in the treated individual and the control individual(s) are comparable).

Subject: As used herein, the term “subject”, “individual”, or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.). In some embodiments, the subject to be treated is an individual (infant, child, adolescent, or adult human) having or having the potential to develop overweight, obesity, diabetes, or gastrointestinal dysbiosis. In some instances, a subject to be treated is genetically predisposed to developing overweight, obesity, diabetes, or gastrointestinal dysbiosis.

Therapeutic agent: As used herein, the phrase “therapeutic agent” refers to any agent that, when administered to a subject, has a therapeutic effect and/or elicits a desired pharmacological and/or biological effect.

Therapeutic regimen: As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of (e.g., across a relevant population of cells, tissues, or organisms) a particular effect, e.g., reduction or elimination of a detrimental condition or disease. In some embodiments, treatment includes administration of one or more therapeutic agents either simultaneously, sequentially or at different times, for the same or different amounts of time.

Therapeutically effective amount: As used herein, the term “therapeutically effective amount” refers to an amount of a therapeutic agent (e.g., an edible fiber comprising glyceollins) which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the particular form of overweight, obesity, diabetes, IBD, or gastrointestinal dysbiosis being treated; the severity of the condition or pre-condition: the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

Treatment: As used herein, the term “treatment” (also “treat” or “treating”) refers to any administration of a therapeutic agent (e.g., an edible fiber comprising glyceollins) according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition (e.g., overweight, obesity, prediabetes, diabetes, gastrointestinal dysbiosis); in some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION

The present disclosure encompasses the findings that edible fiber can be produced from soy plant tissue and food products containing edible fiber enhanced with glyceollins is useful for the treatment or prevention of overweight, obesity, prediabetes, diabetes, IBD, and gastrointestinal dysbiosis.

### Edible Fiber Produced from Soy Plant

Most soy products are prepared from soybeans (the soy seeds contained in the soy pod). Those products are derived from soy oil and soy protein in the soybeans. Common products are soy sauce, soy oil, soymilk, and tofu. The shell of the soybean pod is the ovary wall. This protects the ovules (seeds or beans) and provides a safe environment for them to grow and mature. Soy pods are dehiscent, meaning they have a seam that runs along both sides that can split open. The inside of a soy pod is known as the locule. Other than edamame, there are currently no edible products produced from the pod. Further, most consumers do not eat the pods when served unshelled edamame (pods with beans). It is more popular to serve shelled edamame or the soybeans, which are eaten uncooked or after cooking. Edamame is a variety of soy that is engineered or selected to offer tasty large beans when picked during the middle stages of the bean growth and maturity. However, for the production of edible fiber as described herein, any variety of soy can be used when the bean is harvested at a middle reproductive stage.

### Enhancing Glyceollin Content of Soy Plant Tissue

The soy pod does not synthesize glyceollin unless it is exposed to an environmental stressor. Exposure to UV light offers an efficient elicitor of glyceollin by increasing the expression of polyphenylalanine ammonia-lyase and chalcone synthase. UV photoactivation lends itself to large-scale low cost development of a marketed product. Additionally, or alternatively, glyceollin may be elicited by slicing.

Soy pods will typically be harvested at reproductive stage R6. This stage contains green seeds that fill the pod cavity, touch both sides of the pod, and is the stage that edamame is harvested. This is a good source of edible fiber because pods contain bioactive isoflavones and they contain a blend of soluble and insoluble fiber. Moreover, soy pods are capable of producing the glyceollins like the seeds.

The entire pod with bean may be processed-into small cross-sections or slices by a food processor. In other instances, the pods maybe opened to harvest the beans and the pods can be sliced into small sections with a food processor.

Pods, cut pods, or sliced pods can be placed on wet filter paper and incubated in a humidified atmosphere (85% humidity) in the dark for 48-96 h to permit the glyceollins to be synthesized and to accumulate in the sections. Glyceollin can also be stimulated by irradiation using an ultraviolet (UV) light system producing UV-B light. Plant tissue can be arranged to expose one surface facing the lamp for 30-120 seconds and the tissue will be inverted to expose the other side for an additional 30-120 seconds. Photoactivated soy pod tissues can be placed at room temperature in a humidified chamber (83% humidity) in the dark for 24-72 h to permit the glyceollins to accumulate.

Soy isoflavones can be extracted with methanol and analyzed by HPLC to measure the extent of photoactivation. Daidzin, genistin, malonyldaidzin, malonylgenistin, daidzein, genistein, coumestrol, glyceollin III, glyceollin II, and glyceollin I can be measured.

If the desired isoflavone content of the activated fiber is not achieved under optimal conditions, an aliquot of the blend can be extracted with ethanol to concentrate the isoflavones into a stock solution so that the fiber can be spiked to contain the desired target content of isoflavones in the powdered fiber.

### Production of Powder

Edible or dietary fiber is defined as the remnants of plant components resistant to hydrolysis by human alimentary enzymes which include non-starch polysaccharides, resistant starch and lignin. Edible fiber is typically isolated from oats, barley, chicory roots, and sugar beets. Prior to this disclosure, dietary fiber has not been developed from soy pods. The plant material after the slicing stress is dried using a freeze dryer for 24-120 hours. The dried material can then be ground to a fine powder using a mill with a screen sifting and collecting particles between less than 0.5 mm. This milled material contains both soluble and insoluble fibers.

### Oral Administration

It is preferred that the compositions described herein be consumed orally so that the fiber enters the digestive tract. That will permit the fiber to interact with the microbiota that are resident in the lower GI tract. In addition, the soluble fiber will contribute to increased residence time in the colon for the insoluble fiber as well as the isoflavones. Some of the bacteria will thrive on the fiber and produce healthy byproducts such as small chain fatty acids that can be absorbed into the blood, serve as nutrients for the intestines, and serve as substrates for other bacteria. The novel fiber will also alter the redox potential of the intestinal milieu, which will aid in selection of desired species of healthy microbiota and in shifting the GI microbiome from an unhealthy state to one promoting heath.

Select diets, foods, food ingredients and other compositions comprising glyceollins all have the potential to interact and modify the GI microbiome if they are ingested, not metabolized by the digestive system and not absorbed by the intestines.

A therapeutically effective amount of the compositions described herein is largely determined based on the total amount of edible fiber and/or glyceollins contained in the food products described herein. Generally, a therapeutically effective amount is sufficient to achieve a meaningful benefit to a subject (e.g., treating, modulating, curing, preventing and/or ameliorating overweight, obesity, diabetes, or gastrointestinal dysbiosis).

In some embodiments, a therapeutically effective amount ranges from about 0.005 mg/kg body weight to 15 mg/kg body weight, e.g., from about 0.005 mg/kg body weight to about 12 mg/kg body weight, from about 0.005 mg/kg body weight to about 10 mg/kg body weight, from about 0.005 mg/kg body weight to about 5 mg/kg body weight, from about 0.005 mg/kg body weight to about 1 mg/kg body weight, from about 0.01 mg/kg body weight to about 15 mg/kg body weight, from about 0.01 mg/kg body weight to about 10 mg/kg body weight, from about 0.01 mg/kg body weight to about 5 mg/kg body weight, from about 0.01 mg/kg body weight to about 1 mg/kg body weight, from about 0.1 mg/kg body weight to about 15 mg/kg body weight, from about 0.1 mg/kg body weight to about 10 mg/kg body weight, from about 0.1 mg/kg body weight to about 2 mg/kg body weight, from about 0.1 mg/kg body weight to about 1 mg/kg body weight, from about 1 mg/kg body weight to about 15 mg/kg body weight, from about 5 mg/kg body weight to 15 mg/kg body weight, or from about 5 mg/kg body weight to 10 mg/kg body weight.

In some embodiments, a therapeutically effective dose is greater than about 0.0001 mg/kg body weight, greater than about 0.0005 mg/kg body weight, greater than about 0.001 mg/kg body weight, greater than about 0.005 mg/kg body weight, greater than about 0.01 mg/kg body weight, greater than about 0.05 mg/kg body weight, greater than about 0.1 mg/kg body weight, greater than about 0.5 mg/kg body weight, greater than about 1 mg/kg body weight, greater than about 5 mg/kg body weight, greater than about 10 mg/kg body weight, or greater than about 15 mg/kg body weight.

In some embodiments, a therapeutically effective dose can be expressed as an amount per unit volume. It is to be further understood that for any particular subject, specific dosage regimens can be adjusted over time according to the individual need and that dosage ranges set forth herein are exemplary only and are not intended to limit the scope or practice of the claimed invention.

### Therapeutic Uses

The present invention encompasses the surprising finding that oral administration of food products comprising glyceollins are useful, among other things, in the treatment or prevention (i.e., delay of onset) of overweight, obesity, prediabetes, diabetes. IBD, and gastrointestinal dysbiosis.

### Treatment of Overweight or Obesity

In certain embodiments, treatment of overweight or obesity refers to partial or complete alleviation, amelioration, relief, inhibition, delaying onset, reducing severity and/or incidence of symptoms.

Obesity is a complex, multi-factorial chronic disease involving environmental (social and cultural), genetic, physiologic, metabolic, behavioral and psychological components. It is the second leading cause of preventable death in the United States. Obesity increases the risk of developing hypertension, type 2 diabetes, stroke, gallbladder disease, infertility, osteoarthritis, sleep apnea, and cancer of the breast, prostate and colon. Persons with obesity may also be victims of employment and other discrimination and are penalized for their condition despite many federal and state laws and policies.

If maintained, even weight losses as small as percent of body weight can improve the risk of developing the above diseases. In particular, hypertension, control of blood glucose and sleep apnea are improved with fat loss.

One goal in obesity treatment is to reduce excess fat storage. More specifically, to reduce extra-adipose fat stores. This may be measured by instruments using x-ray technologies, magnetic resonance technologies, and volume displacement technologies. More simply, it may be estimated simply by measuring body weight, skin fold thickness and waist circumference. Sometimes an index of improvement is observed by changes in biomarkers such as a decrease in blood lipids, increased insulin sensitivity, decrease in circulating liver enzymes, decrease in leptin, increase in adiponectin and a decrease in markers of inflammation.

### Treatment of Diabetes and Related Disorders

In certain embodiments, treatment of diabetes or prediabetes refers to partial or complete alleviation, amelioration, relief, inhibition, delaying onset, reducing severity and/or incidence of symptoms.

Glucose comes from the food and is also produced by liver and skeletal muscles where it is stored as glycogen. Insulin is a hormone, made by the pancreas and is released into the blood when glucose levels rise. Insulin transports glucose from the blood into cells of tissues to be used for energy. If insulin levels released are too low, or if the cells are resistant to insulin, glucose can't enter certain cells and remains in the blood. Blood glucose levels rise and are used to diagnose prediabetes or diabetes. Prediabetes is defined by the American Diabetes Association as fasting blood glucose levels between 100 mg/dl and 125 mg/dl, or blood glucose level between 140 mg/dl and 125 mg/dl 2 h after an oral glucose tolerance test (OGTT) and a hemoglobin A1c level between 5.7% and 6.4%.

Type 1 diabetes, formerly called juvenile diabetes or insulin-dependent diabetes, is usually first diagnosed in children, teenagers, or young adults. With this form of diabetes, the pancreas no longer makes insulin because the body's immune system has attacked and destroyed the insulin producing cells. Treatment for type 1 diabetes includes insulin injections.

Type 2 diabetes, formerly called adult-onset diabetes or noninsulin-dependent diabetes, is the most common form of diabetes. People can develop type 2 diabetes at any age-even during childhood. This form of diabetes usually begins with insulin resistance, a condition in which fat, muscle, and liver cells do not respond to insulin properly. At first, the pancreas keeps up with the added demand by producing more insulin. In time, however, it loses the ability to secrete enough insulin in response to meals. Being overweight and inactive increases the chances of developing type 2 diabetes.

Some women develop gestational diabetes during the late stages of pregnancy. Although this form of diabetes usually goes away after the baby is born, a woman who has had it is more likely to develop type 2 diabetes later in life.

Symptoms of diabetes include increased thirst, frequent urination, frequent infections, blurred vision, feeling tired, slow wound healing, tingling and (or) numbness in the hands and (or) feet, and recurring skin, gum, or bladder infections, weight loss, nausea, and vomiting. If not treated the patients are at greater risk for many additional ailments.

People with diabetes are at increased risk for eye complications such as retinopathy. Diabetics are also at increased risk of nerve damage. Ulcers occur most often on the ball of the foot or on the bottom of the big toe. As many as 2 out of 3 adults with diabetes have high blood pressure. Hearing loss is twice as common in people with diabetes as it is in those who don't have the disease. Research shows that there is an increased prevalence of gum disease among those with diabetes. Gastroparesis is a disorder affecting people with both type 1 and type 2 diabetes in which the stomach takes too long to empty its contents (delayed gastric emptying). Diabetes can damage the kidneys and cause them to fail. Two out of 3 people with diabetes die from stroke or heart disease.

Improvement in diabetes is typically measured by analyzing blood glucose levels during fasting, after meals, after ingestion of a glucose drink and before bedtime. Lower fasting glucose levels and a more rapid and complete return to baseline glucose values after a meal or oral glucose challenge serve as indications of improvement.

### Treatment of Inflammatory Bowel Disease

In certain embodiments, treatment of IBD refers to partial or complete alleviation, amelioration, relief, inhibition, delaying onset, reducing severity and/or incidence of symptoms. IBD is a complex, multi-factorial chronic disease involving environmental (social and cultural), genetic, physiologic, metabolic, behavioral and psychological components. IBD can be chronic or recurring. It is an immune response leading to inflammation of the gastrointestinal (GI) tract. The two most common inflammatory bowel diseases are ulcerative colitis (UC) and Crohn's disease (CD). More than 1.5 million Americans have Crohn's disease or ulcerative colitis.

IBD can be painful and debilitating, and sometimes leads to life-threatening complications.

Symptoms usually develop over time, rather than suddenly. UC usually affects only the innermost lining of the large intestine (colon) and rectum. Crohn's disease causes inflammation anywhere along the lining of your digestive tract, and often spreads deep into affected tissues. This can lead to abdominal pain, severe diarrhea and even malnutrition. The inflammation caused by Crohn's disease can involve different areas of the digestive tract in different people.

The goals of IBD therapy are to eliminate symptoms, prevent flare-ups (maintain long-term remission) and restore quality of life. For most people, medications control symptoms and promote healing. These include antibiotics, anti-inflammatory steroids, and biologics such as antibodies to cytokines such as IL-10. Surgery is usually needed only if medications fail to improve symptoms or if precancerous changes in the colon or serious complications occur.

### Treatment of Gastrointestinal Dysbiosis

In certain embodiments, treatment of gastrointestinal dysbiosis refers to partial or complete alleviation, amelioration, relief, inhibition, delaying onset, reducing severity and/or incidence of symptoms.

The GI microbiome may be characterized in healthy individuals and those inflicted with disease. In healthy individuals the GI microbiome is defined as normal. The GI microbiome characterized in those with certain diseases such as diabetes, obesity, irritable bowel syndrome (IBS) and irritable bowel disorder (IBD) are referred to as being in a state of dysbiosis. Currently, the symptoms and consequences of the pathological states define the diseases. It is unknown to what extent the dysbiosis contributes to the pathology or to what extent the dysbiosis is a consequence of that pathology. Nonetheless, the pathology or consequences thereof may be treated by converting the dysbiosis back to a normal GI microbiome.

GI dysbiosis is typically characterized as the microbiota community in a stool sample of an individual in a pathological state. In some cases, the dysbiosis results in reduced levels of SCFAs in the stool, increased fecal pH, increased production of hydrogen sulfide and methane gases, reduced antioxidant capacity, presence of opportunistic microbiota, presence of pathogenic fungi and yeast, increased intestinal inflammation, decreased intestinal mucosal thickness, colon ulcers and leaky gut. Improvements may be observed from increased SCFA levels in stool, decreased fecal pH, decreased production of hydrogen sulfide and methane gases, increased antioxidant capacity, absence of opportunistic microbiota, absence of pathogenic fungi and yeast, decreased intestinal inflammation, normal intestinal mucosal thickness, healthy colon anatomy and less circulating immunoglobulin A antibodies.

### Examples

The invention is further illustrated by the following examples. The examples are provided for illustrative purposes only. They are not to be construed as limiting the scope or content of the invention in any way.

### Example 1

### Glyceollin Bioavailability

Male ZDSD/Pco rats were bred onsite at PreClinOmics (PreCclinOomics, Indianapolis, Ind.), individually housed in suspended wire cages, and maintained on a 12:12 hour light-dark cycle under standard laboratory conditions with a controlled room temperature (20-21° C.). The protocol and all procedures were approved by the Institutional Animal Care and Use Committee of PreClinOmics approved the protocol and all procedures. Rats with a 3-month bodyweight of approximately 500 g were chosen for the experiments since they would not yet have developed diabetes. Diabetic synchronization can be achieved by feeding a calorie dense diet. However, because a prediabetic model was needed for this study, the ZDSD/Pco rats received ground irradiated Purina 5008 chow (Ralston Purina, Belmont, Calif.) to maintain a prediabetic state throughout the study. Chow was placed in spill resistant jars for accurate food intake measurements and the rats had free access to drinking water.

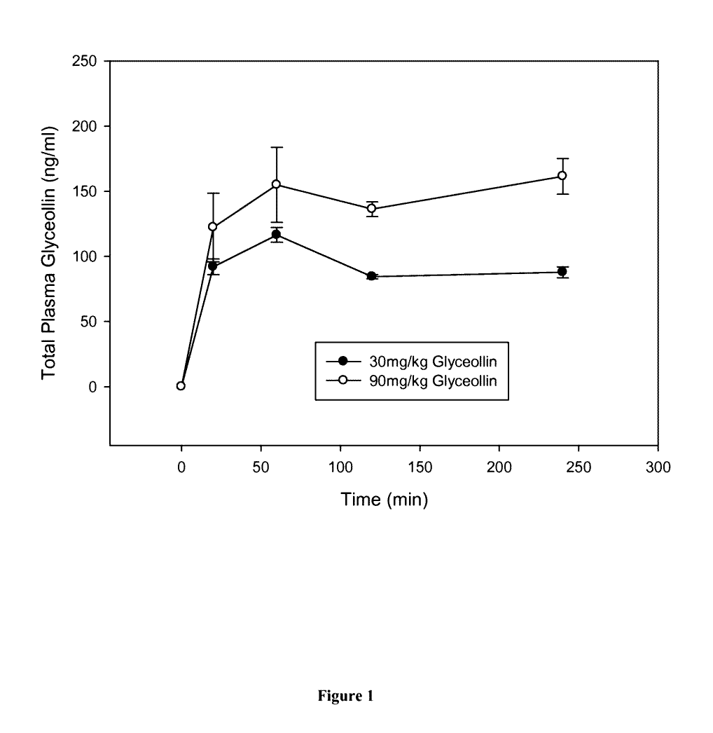

Glyceollins were administered via oral gavage (3 mL) to rats in the fed state. The study design included the following groups (n=3 rats per group): vehicle (poloxamer 407; 7.5% in water), glyceollins dissolved in poloxamer to administer 30 mg/kg and 90 mg/kg. Blood levels of glyceollins were measured 0.5-, 1-, 2-, and 4-h after oral gavage. The animals were euthanized by decapitation and trunk blood was collected into EDTA coated tubes supplemented with aprotinin. Plasma was separated and stored at −80° C. until analysis by HPLC-ESI-MS/MS (FIG. 1). These data demonstrate that glyceollins are absorbed after oral administration into the circulation to some extent, exposing cells to the isoflavones. The plasma levels were low but sustained for 4 h. The data also suggest glyceollins that are not absorbed remain in the GI tract.

### Example 2

### Oral Glucose Tolerance Test

Male ZDSD/Pco rats were bred onsite at PreClinOmics (PreCclinOomics, Indianapolis, Ind.), individually housed in suspended wire cages, and maintained on a 12:12 hour light-dark cycle under standard laboratory conditions with a controlled room temperature (20-21° C.). The protocol and all procedures were approved by the Institutional Animal Care and Use Committee of PreClinOmics approved the protocol and all procedures. Rats with a 3-month bodyweight of approximately 500 g were chosen for the experiments since they would not yet have developed diabetes. Diabetic synchronization can be achieved by feeding a calorie dense diet. However, because a prediabetic model was needed for this study, the ZDSD/Pco rats received ground irradiated Purina 5008 chow (Ralston Purina, Belmont, Calif.) to maintain a prediabetic state throughout the study. Chow was placed in spill resistant jars for accurate food intake measurements and the rats had free access to drinking water.

Eight rats were randomly assigned to receive either glyceollins (30 mg/kg or 90 mg/kg) or vehicle at the onset of the photoperiod dark cycle as described in Example 1. An oral glucose tolerance test (OGTT) was performed as described below on dl of treatment.

After fasting for 5 h into the dark cycle of the photoperiod, glyceollins were administered via oral gavage as described above. There were 8 rats in each group for this experiment. On the 6^{th} h. the rats were dosed with glucose (2 g/kg, 10 ml/kg, p.o.). Tail vein blood was sampled for glucose measurement at −15-, 30-, 60-, 90-, and 120-minutes after the glucose challenge. Whole blood glucose levels were measured using an AlphaTrak blood glucose monitor (Abbott Laboratories, Abbott Park, Ill.).

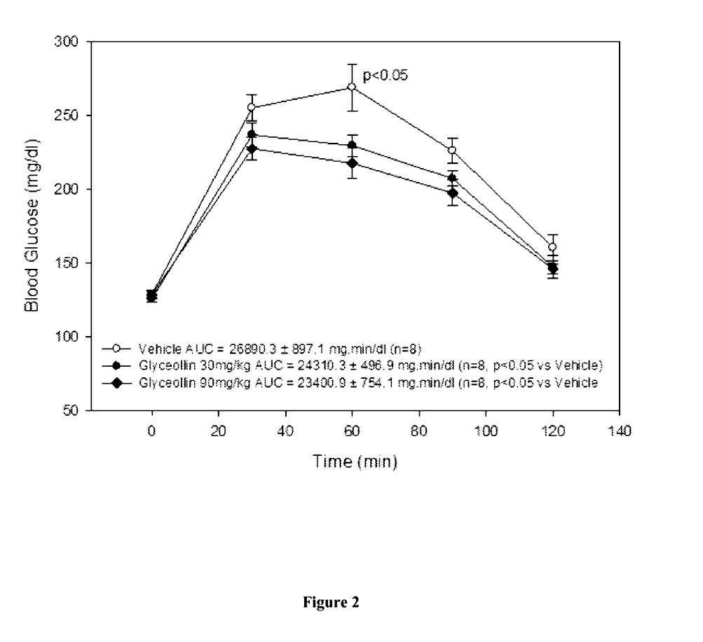

The ZDSD/Pco rats were in a prediabetic state as evidenced by the fasting blood glucose value of 127.6±1.5 mg/dl (n=24). Blood glucose increased to a maximum level at 30 min after the oral glucose gavage, it remained elevated in the vehicle group until 60 min but was significantly (p<0.05) less at that time in both glyceollin groups (FIG. 2). Disposal of the circulating glucose during the 120 min period of the oral challenge was significantly (p<0.05) greater in both glyceollin groups than in the vehicle group (AUC for the OGT was 26890±876, 24310.496, and 23401±754 mg/min/dl, for vehicle group (n=8), 30 mg/kg glyceollin group (n=8) and 90 mg/kg glyceollin group (n=8). There was no significant difference between the 2 glyceollin groups.

The data demonstrate that pretreatment with a mixture of 3 glyceollins improved the blood glucose response of prediabetic ZDSD rats to an oral glucose challenge.

### Example 3

### Activity in Adipocytes

To determine if one mechanism for glyceollins to improve the oral glucose challenge in a prediabetic model is by increasing the glucose uptake by fat, glyceollin pharmacology was studied in 3T3-L1 cells. Murine preadipocytes (Zen-Bio Inc.) were cultured using PM-1-L1 medium (Zen-Bio Inc.) containing Dulbecco's modified Eagle's medium (DMEM)/Ham's F-10 medium (1:1, v/v), HEPES 15 mM (pH 7.4), 10% (v/v) fetal bovine serum, penicillin (100 U/ml), streptomvcin (100 mg/ml), and amphotericin B (0.25 μg/ml) in a humidified atmosphere (5% CO₂/95% air). After 3-4 days, confluent cells were placed in differentiation medium (DM-2-L1, Zen-Bio Inc.) containing DMEM/Ham's F-10 medium (1:1, v/v), HEPES 15 mM (pH 7.4), 3% (v/v) fetal bovine serum, biotin (33 M), pantothenate (17 μM), human insulin (100 nM), dexamethasone (1 μM), penicillin (100 U/ml), streptomycin (100 μg/ml), amphotericin B (0.25 μg/ml), isobutylmethylxanthine (0.20 μM) and PPARγ agonist (10 μM) and further incubated in the humidified atmosphere for 3 days. The medium was then changed to AM-1-L1 medium (Zen-Bio Inc.) containing DMEM/Ham's F-10 medium (1:1, vi/v), HEPES 15 mM (pH 7.4), 3% (v/v) fetal bovine serum, biotin (33 μM), pantothenate (17 μM), human insulin (100 nM), dexamethasone (1 μM), penicillin (100 U/ml), streptomycin (100 μg/ml), and amphotericin B (0.25 μg/ml). AM-1-L1 medium (i.e., adipocyte maintenance medium, such as those commercially provided by ZenBio©) was changed every 2-3 days during an additional 10 days of incubation.

Adipocytes were rinsed in sterile, fresh KRH buffer (HEPES pH=7.4, 1 mM CaCl₂, 1.2 mM MgSO₄, 1 mM KH₂PO₄, 1.4 mM KCl, 20 mM, 130 mM NaCl), and then preincubated for 24 h in KRH buffer. The buffer was removed, and adipocytes were incubated in KRH buffer containing glyceollins (at concentrations indicated, such as at 0.5 μM-5 μM) for the specified time period. 10 μL of [³H]-2-deoxy-D-glucose (Vitrax, Placentia, Calif.) diluted to 0.01 μCi/μL with D-glucose (100 mM) was added to each well and incubated 10 min in a 37° C. water bath. The supernatant was removed, and plates were rinsed rapidly three times with ice cold KRH. The final rinse was aspirated, taking care not to remove the cellular monolayer, then 500 μL ice cold RIPA buffer (Sigma-Aldrich, St. Louis. Mo.) was added to lyse the cells. The cellular content in each well was triturated with a 1 ml pipette several times to remove attached cells and cellular components from the bottom of the plate. Aliquots of 450 μL were transferred to vials containing 5 mL Ecolume scintillation fluid (MP Biomedical. Santa Ana, Calif.). The vials were mixed and counted for 10 min in an Applied Biosystems 1100 liquid scintillation counter using the factory preset window to detect tritium.

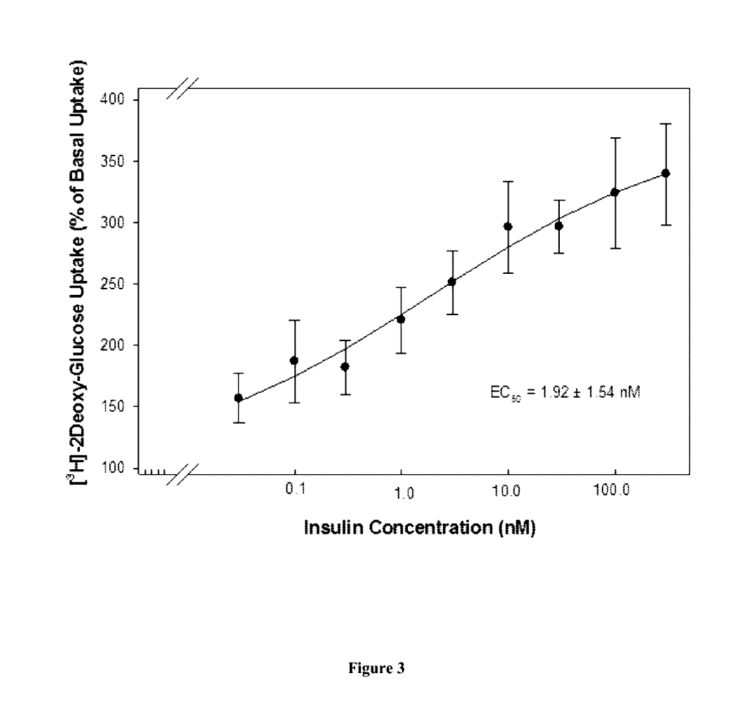

These adipocytes respond well to insulin stimulation (FIG. 3). Glucose was transported into the adipocytes in a dose-dependent manner (0.3 nM-300 nM insulin). Maximal stimulation was observed to be about 3-times that measured for basal glucose uptake and the concentration of insulin that produced half of that response (EC₅₀) was calculated to be 1.9±1.5 nM (n=6).

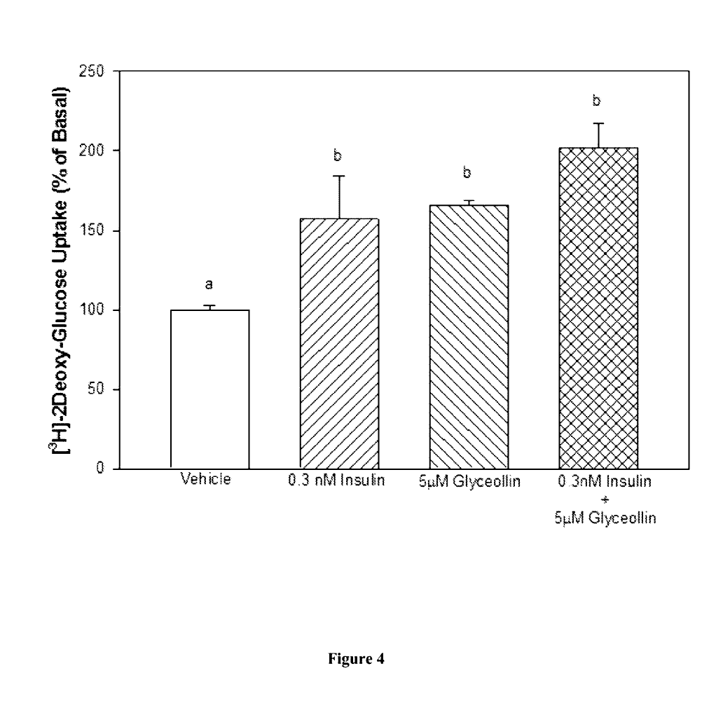

To determine whether the response of adipocytes to insulin is potentiated by glyceollins, 3T3-L1 differentiated cells were incubated with DMSO (vehicle control), 0.3 nM insulin, 5 μM glyceollins, or both glyceollin mix with insulin. Although the glucose uptake stimulated by insulin with glyceollins tended to be greater, the increase was not significantly different (FIG. 4). Surprisingly, the glyceollin blend was as efficacious as insulin in stimulating glucose uptake but less potent.

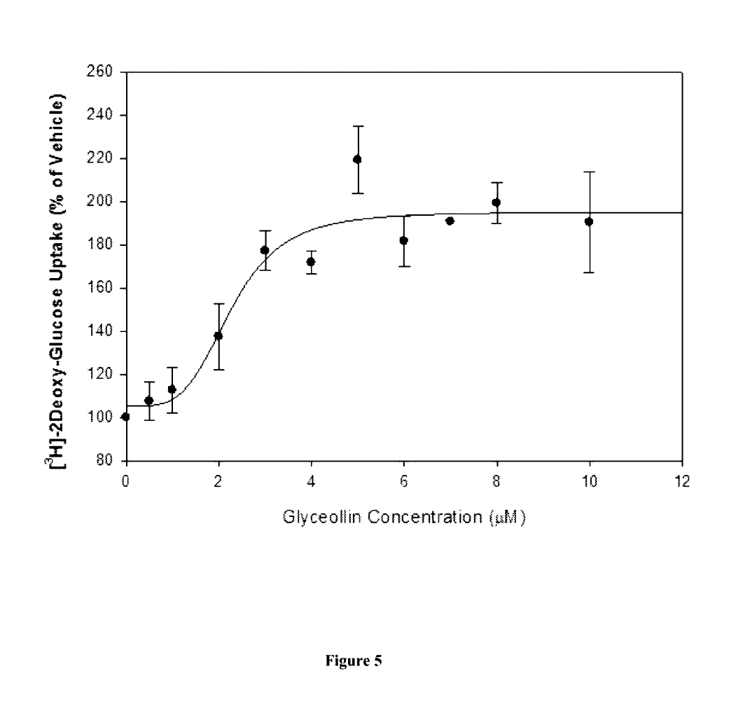

In order to study the dose-glucose uptake response to glyceollin, dose ranging studies were performed (FIG. 5). Glucose uptake was stimulated by 45 min exposure to glyceollins at doses ranging between 0.5 μM and 10 μM with an EC₅₀ of 2.40±0.43 μM and a maximal uptake of 2.04±0.24-fold stimulation above basal glucose uptake (n=3).

To determine the underlying mechanism for glyceollin stimulation of glucose uptake, the expression of the genes encoding GLUT1 and GLUT4, which are the key glucose transporter protein genes expressed in adipocytes, was examined.

Adipocytes were grown in 6-well plates, as described above, and used at day 10-11 after initiation of differentiation. Adipocytes were rinsed in sterile KRH buffer, and then preincubated for 24 h in KRH buffer. The buffer was removed and adipocytes were treated with either DMSO as a vehicle, or glyceollins (at concentrations indicated, such as 1 μM or 10 μM) for 3 h. Total RNA was isolated using Trizol reagent (Invitrogen) and purified on RNeasy columns (Qiagen) according to the manufacturer's protocol. RNA quality and concentration was determined by absorbance at 260 nm and 280 nm. Total RNA was reverse-transcribed using a QuantiTect Reverse Transcription kit (Qiagen). The sequences of the forward primer, reverse primer, and TaqMan probes for GLUT1, GLUT4, and the housekeeping gene ribosomal protein L32 (RPL32) (NM_172086) are described, in Cao, H. et al., Obesity 16:1208-1218 (2008). The reactions were performed in 96-well plates in a CFX96 Real-Time PCR Detection Systems (Bio-Rad). The thermal cycle conditions were as follows: 2 min at 50 C and 10 min at 95 C. followed by 50 cycles at 95 C for 15 s each and 60° C. for 60 s. The ΔΔC_{T} method of relative quantification was used to determine the fold change in expression. This was done by first normalizing the resulting threshold cycle (C_{T}) values of the target mRNAs to the C_{T} values of the internal control Rpl32 in the same samples. Those data were compared to the DMSO control

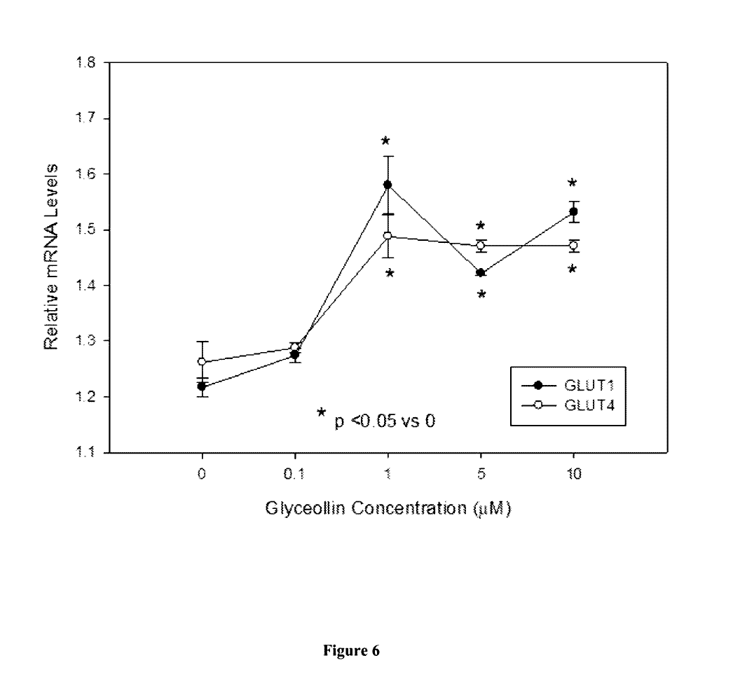

Expression of both GLUT1 and GLUT4 were significantly increased in cells exposed to glyceollin ranging from 1 μM-10 μM (FIG. 6).

These findings establish that a mechanism for glyceollin-mediated glucose uptake into fat cells is by increasing the expression of both GLUT1 and GLUT4 genes. GLUT1 is thought to be responsible for basal glucose uptake by adipocytes and most other cells. GLUT4 is also expressed by adipose and other insulin target tissues. It is thought to be responsible for insulin-stimulated glucose uptake. Both GLUTs are expressed by 3T3-L1 cells after they differentiate into mature adipocytes. Thus, that glyceollins may act in concert with insulin or independently of the hormone to stimulate glucose uptake by adipocytes.

### Example 4

### Chronic Administration of Glyceollin to Prediabetic Rats

A blend of the 3 glyceollins (glyceollin I, glyceollin II and glyceollin III) to prediabetic ZDSD/Pco rats improves the blood glucose response to an oral glucose challenge (see Example 2). The pharmacokinetic data also demonstrated that glyceollin is only partially bioavailable after oral administration since plasma levels during 3 h after administration of either 30 mg/kg or 90 mg/kg were low (see Example 1). A study was performed using ZDSD/Pco rats to determine if oral administration of the glyceollins alters the GI microbiome and body composition.

The rats were treated with oral doses of the glyceollin blend (90 mg/kg) for 11 d and the microbiota taxa in feces was analyzed before treatment and on day 11 of treatment. A Beckman Synchron CX4 random-access multianalyzer (Beckman Coulter. Inc., Brea, Calif.) was used to measure glucose (cat # OSR6121), cholesterol (cat # OSR6116) and triglycerides (cat # OSR6018) in plasma at the terminal bleed. Active GLP-1 and insulin were measured using a Meso Scale Discovery multiplex instrument and the K11159c-1 kit (Meso Scale Discovery, Inc., Gaithersburg, Md.). Leptin was measured by ELISA using an ALPCO Immunoassay rat/mouse leptin kit (22-LEPMS-E01).

Differences in abundance of 3 genera were observed when comparing baseline to the microbiota signature of feces obtained on day 11 of glyceollin treatment (Table 1). In particular, there was a dramatic bloom observed in species of Blautia. No shift in diversity or abundance was observed in taxa from vehicle treated rats when comparing fecal microbiota at pretreatment with that from treatment d11.

TABLE 1
Significant changes in 3 genera of microbiota after
only 11 days of treatment with a glyceollin blend.

| | Pretreatment | Day 11 | |
|---|---|---|---|
| | Abundance¹ | Abundance | |
| | (% of total, | (% of total, | |
| Genus | mean ± SEM) | mean ± SEM) | Probability |
| Blautia | 9.70 ± 0.56 | 20.68 ± 2.21 | 0.0005 |
| Peptostreptococcaceae | 0.00 ± 0.00 | 3.60 ± 1.34 | 0.01 |
| Ruminococcaceae | 3.87 ± 0.58 | 1.91 ± 0.54 | 0.02 |

¹Analytical data are from MultiTag Sequencing that was performed by Metabiomics (http://metabiomics.com/services/).

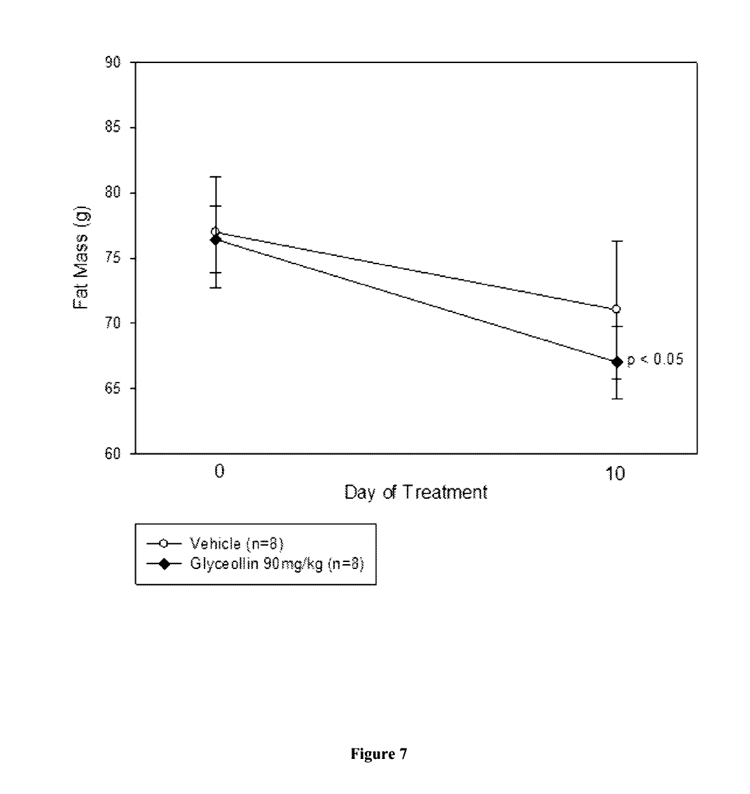

This change in the fecal profile of microbiota was significantly correlated (p<0.05) to a decrease in body weight (from 529.25±8.15 g to 529.00±10.32 g, vehicle group; from 529.88±5.84 to 523.88±6.44 g, glyceollin blend) that was primarily a consequence of decreased fat mass (FIG. 7).

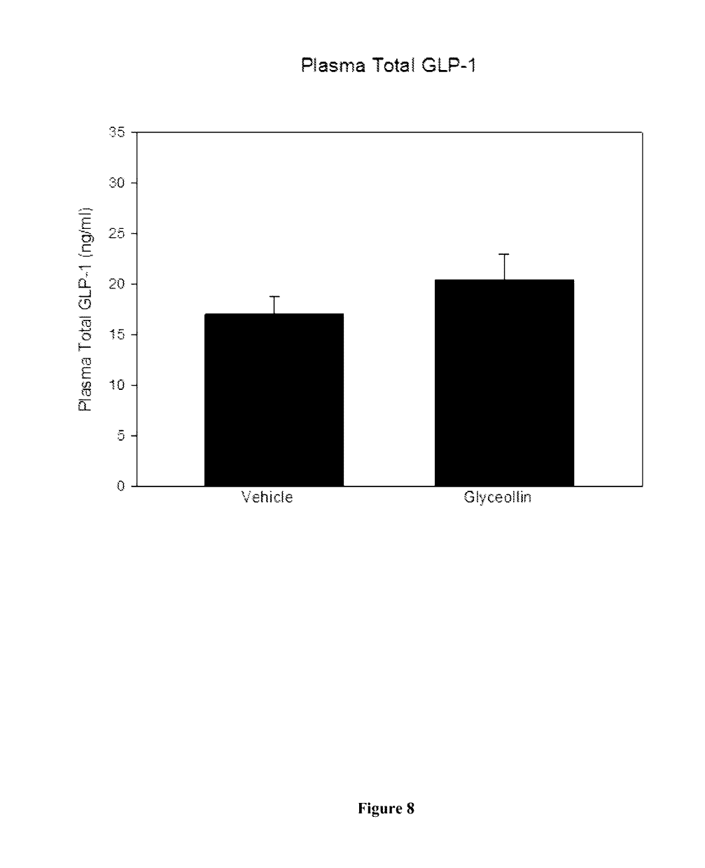

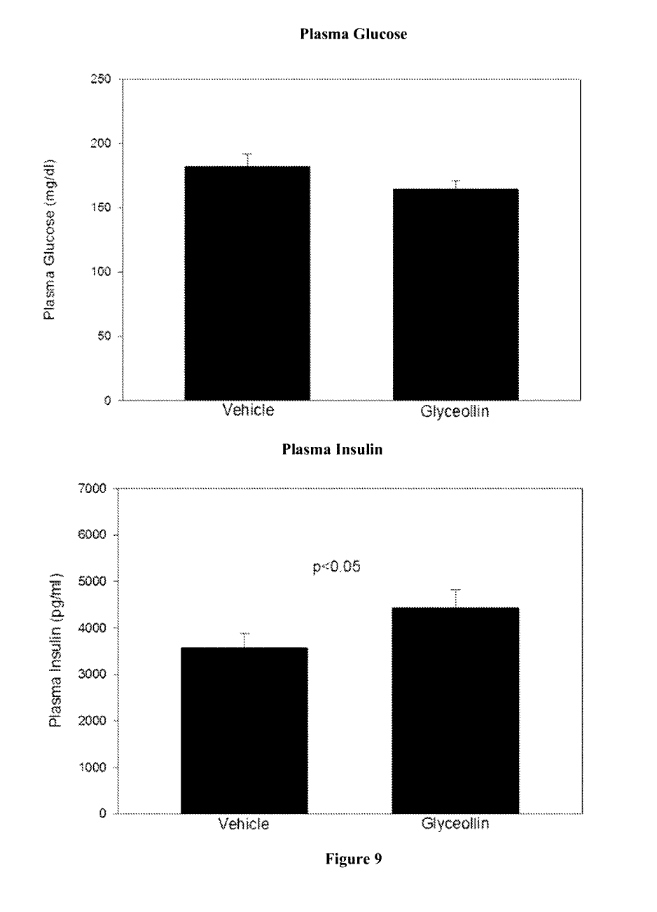

The shift in fecal microbiota profile was also significantly correlated (p<0.05) to decreases in plasma leptin as well as increases in plasma GLP-1 (FIG. 8) and plasma insulin (FIG. 9).

The glyceollin blend stimulated a dramatic bloom in species of Blautia after just 11 d of treatment. Species of this genus are hydrogen-consuming organisms that also have genes indicating that they can process polyphenolic molecules and can synthesize acetate (Liu et al., 2008, Int. J. Syst. Evol. Microbiol. 58:1896-902). Dietary components such as fiber that reach the colon is fermented principally to SCFAs, but hydrogen and carbon dioxide are also generated in that process. Microbial disposal of the hydrogen generated during anaerobic fermentation in the human colon is important for optimal functioning of this ecosystem (for review see Nakamura et al. 2010; Annu. Rev. Food Sci. Technol. 1:363-95). There are 2 other major groups of hydrogen-consuming microorganisms found in the colon, the methanogens and sulfate reducing bacteria. Both appear to occur mainly for hydrogen utilization and are in competition with each other as well as with the species of Blautia.

In a simplified model of human gut community relationships, transplantation of germ-free mice with Bacteroides thetaiotaomicron (capable of fermenting carbohydrate to SCFAs and hydrogen) and Methanobrevibacter smithii (capable of utilizing hydrogen and carbon to produce methane) but not a colonization of B. thetaiotaomicron with Desulfovibrio piger (capable of reacting hydrogen with sulfur to produce hydrogen sulfide), resulted in increased serum acetate levels, increased liver triglycerides, and increased adiposity (Samuel and Gordon 2006; Proc Natl Acad Sci. USA; 103:10011-10016). Transplanting Bacteroides thetaiotaomicron with Blautia hydrogenotrophica (capable of reacting hydrogen with carbons from fermentation to produce acetate) results in much greater circulating acetate levels than cotransplantation of B. thetaiotaomicron with M. smithii (Bain et al. 2010 J Biol Chem; 285: 22082-22090). Unfortunately, resulting liver triglycerides and mass of fat depots were not reported in that colonization pairing study. However, these data suggest that one type of hydrogen utilizing bacteria is more conducive for the host to accrue calories and the others are best at producing either acetate or hydrogen sulfide (often associated with colon pathology) when coupled with a carbohydrate fermenting bacteria. Without wishing to be bound by theory, it is believed herein that glyceollin-stimulated bloom in Blautia creates competition between the other 2 classes of hydrogen-consuming microbiota in the gut for hydrogen. Consequently, greater acetate levels in the colon that serve as ligands for satiety hormones as well as serve to generate an inactive ghrelin will induce decreased adipocity. Indeed, like hydrogen, the glyceollins are strong reducing agents and unlike the methanogens or sulfate reducing bacteria. Blautia are capable of processing molecules like polyphenolics (Bain et al. 2010 J Biol Chem; 285: 22082-22090).

### Example 5

### Human Study Utilizing Activated Soy Pod Fiber to Correct the GI Dysbiosis Observed in Type 2 Diabetes, Improve Glucose Regulation and Improve Body Composition

### Subjects and Methods

The required number of subjects are properly screened to fulfill the necessary qualifications, appropriate laboratory evaluations are performed, measures of positive primary and secondary outcome responses are recorded, adverse events are documented, and patients are adequately followed-up.

### Overview

This is designed to exemplify that overweight subjects with impaired fasting blood glucose on an ad libitum diet who take Activated Soy Pod Fiber either within 1 hour prior to meal 1 or within 1 hour prior to meal 2, as well as within 1 hour prior to meal 3 for 4 weeks, will:

1. Eliminate stool with an increased small chain fatty acids, decreased methane and hydrogen sulfide gases, increased acetate and increased antioxidants when compared to stool analyzed at the start of the intervention, and when compared to subjects consuming a placebo, and
2. Have an improved oral glucose tolerance test (OGTT) as measured by blood glucose and insulin levels before, during, and at 120 minutes after ingestion of 75 g glucose when compared to their initial OGTT, and when compared to subjects consuming placebo, and
3. Have lower overnight fasting blood glucose levels as measured by a blood glucose monitor before ingesting a morning meal when compared to their overnight fasting blood glucose values at the start of the intervention, and when compared to subjects consuming placebo.
4. Experience an improved body composition as measured by a decrease in body weight, a decrease in body fat or % body fat, a decrease in waist circumference measurements, and
5. Experience decreased appetite before a meal, increased satiety during the meal, when compared to subjects consuming placebo, and
6. Will be found to have elevated GLP-1 as well as PYY levels with reduced active ghrelin levels after a standardized meal when the values are compared to those of subjects consuming placebo on week 3 of the intervention.

In this study, subjects consume either 180 ml of Activated Soy Pod Fiber formula or a placebo containing the same total dietary fiber level as Activated Soy Pod Fiber but as inactive cellulose orally within 1 hour prior to consumption of either meal 1 or meal 2 and within 1 hour prior to consumption of meal 3 each day. Placebo formula contains cellulose with food coloring and flavor to match the total dietary fiber content of Activated Soy Pod Fiber. Placebo is prepared by Merlin Development at the same time they prepare Activated Soy Pod Fiber in a palatable easy to mix powder.

Subjects report weekly for measurements and assessment of any side effects. They are asked to collect a stool sample before initiating either Activated Soy Pod Fiber or placebo intervention as well as at the end of the 4-week treatment period. They are also asked to record any side effects and their frequency (checklist assessment). They are asked to record appetite (how hungry are you) and satiety (how full are you) during the standardized meal at the 3^{rd} week of intervention. They are provided with the proper paper work to record these.

### Subject Screening and Selection

A total of 30 subjects is selected, 15 assigned to Activated Soy Pod Fiber and 15 assigned to placebo.

### Qualifications of Subjects

1) Healthy men and women between the ages of 18 and 70 with a BMI between 25 and 45 are eligible. 2) Fasting blood glucose between 100 and 200 mg/dl. 3) Stable weight over 2 months

Subjects Excluded from Study

### People who:

a) take medications affecting glucose,
b) take medications affecting insulin,
c) take medications affecting body weight.
d) take medications affecting bacterial flora.
e) have intestinal disease or a recent history of intestinal disease,
f) have had surgery on stomach or intestine,
g) are hypothyroid,
h) are pregnant,
i) have heart disease.

### Laboratory Evaluation

Different tests are performed at the screening of potential participants, at the beginning of the study, and at the end of the 4-week treatment period.

1) Screening: Subjects are screened to exclude hypothyroidism, pregnancy, and heart disease. The following tests can suffice for this: T4 (thyroxin), T3 (triiodotyronine), TSH (thyroid stimulating hormone), urine pregnancy test, blood pressure & ECG (electrocardiogram).
2) Beginning of study: Subjects passing the initial screen are evaluated at the beginning of Week #1 as follows:
a) Fasting blood glucose and insulin levels
b) SMA 20 (Sequential multi-channel analysis with computer-20, a metabolic panel with 20 different analytes), including, uric acid, and liver function tests
c) Triglycerides
d) Cholesterol, including fractions
e) Glycosylated hemoglobin A1 (HgbA1)
f) Weight, taken on the same scale each time
g) Body fat % and total body fat, determined by DXA (dual-energy X-ray absorptiometry).
h) Height
i) Waist measurements
j) Blood glucose, insulin, GLP-1, PYY and ghrelin responses to a 75 g oral glucose challenge
k) Assessment of appetite and satiety using a visual analog scale
l) Stool is collected and stored frozen but not analyzed until the end of study.
   3) End of study assessment:
a) All labs and assessments done in step 2 at beginning of study,
b) Analysis of the fecal microbiome DNA from both the initial sample and the final sample. c) Analysis of feces that includes: pH, SCFAs, lactoferrin, white blood cells, mucus, secreted immunoglobulin A, anti-gliadin secreted immunoglobulin A, pathogenic bacteria, yeast, fungi, parasites, triglycerides, branched chain fatty acids, long chain fatty acids, and cholesterol.

### Study Design

Subjects selected for participation are allowed an ad libitum diet and are given an evaluation sheet to assess their appetite and satiety before and after a meal. Foods excluded include alcohol. Low calorie liquids are stressed in place of high calorie liquids such as fruit juices, milk, sweet tea (tea with sugar), regular soft drinks, coffee with sugar, etc. The subjects are randomly assigned to either Activated Soy Pod Fiber or placebo treatments. Both the experimenter and the subjects are blinded to who receive Activated Soy Pod Fiber or the placebo. The subjects are encouraged to consume either treatment within 1 hour prior to either breakfast or lunch and within 1 hour prior to dinner.

### Duration

Subjects are given a 4 weeks supply of either Activated Soy Pod Fiber or placebo at the onset and are instructed to consume the entire 180 ml volume within 1 hour prior to either meals 1 or 2, as well as another 180 ml volume containing either Activated Soy Pod Fiber or placebo within 1 hour prior to meal 3. Ad libitum diets are followed for 4 weeks, but the volunteers are instructed to consume either Activated Soy Pod Fiber or placebo as their only between meal snack.

### Outcome

This study is designed to exemplify that Activated Soy Pod Fiber:

1) Eliminate stool with increased small chain fatty acids, decreased methane and hydrogen sulfide gases, increased acetate and increased antioxidants when compared to stool analyzed at the start of the intervention, and when compared to subjects consuming a placebo:

2) Improves the blood glucose and insulin responses to an OGTT by decreasing the areas under the insulin curve (improved insulin sensitivity):

3) Decrease fasting blood glucose values;

4) Produces weight loss, loss of body fat, and (or) decrease of body fat %:

5) Increases GLP-1 and PYY response to the oral glucose challenge and decreases the fasting ghrelin levels at 1 hour after the both the OGTT and the standardized meal when comparing final values to the initial measurements of the OGTT, and when comparing to those findings of the placebo group during the standardized meal:

6) Decreases stool pH;

7) Increases stool SCFA:

8) Increased stool lactoferrin;

9) Decreased stool white blood cells:

10) Decreased stool mucus;

11) Increased stool secreted immunoglobulin A:

12) Increased stool anti-gliadin secreted immunoglobulin A:

13) Decreased stool pathogenic bacteria;

14) Decreased stool yeast, fungi, and parasites;

15) Increased stool triglycerides:

16) Decreased stool branched chain fatty acids;

17) No change in stool long chain fatty acids; and

18) Increased stool cholesterol.

If subjects took Activated Soy Pod Fiber for periods longer than 4 weeks, particularly for at least 8 weeks, the subjects would experience significant weight loss that was primarily fat loss and decreased HgA1c levels.

### Example 6

### Human Study Utilizing Activated Soy Pod Fiber in Combination with an Inhibitor of Dipeptidyl Peptidase-4 (DPP-4) to Correct the GI Dysbiosis Observed in Type 2 Diabetes to and to Improve Glucose Regulation by Sustained Elevation of GLP-1

### Subjects and Methods

The required number of subjects are properly screened to fulfill the necessary qualifications, appropriate laboratory evaluations are performed, measures of positive primary and secondary outcome responses are recorded, adverse events are documented, and patients are adequately followed-up.

### Overview

This study is designed to exemplify that type 2 diabetic (T2D) subjects with insulin resistance on an ad libitum diet who take a DPP-4 inhibitor and Activated Soy Pod Fiber within 1 hour prior to either meal 1 or meal 2, as well as within 1 hour prior to meal 3 for 4 weeks:

1. Eliminate stool characterized as normal at termination of treatment when compared to initiation of treatment and when compared to those only taking the DPP-4 inhibitor, and

2. Have improved insulin sensitivity when compared to both initiation of the study and when compared to those only taking a DPP-4 inhibitor. Insulin sensitivity is measured by an oral glucose tolerance test (OGTT). This is performed by measuring blood glucose and insulin levels before, during, and at 120 minutes after ingestion of 75 g glucose when compared to their initial OGTT, and

3. Have improved fasting blood glucose values when compared to those only taking the DPP-4 inhibitor, and

4. experience an improved body composition as measured by a decrease in body weight, a decrease in body fat or % body fat, a decrease in waist circumference measurements when compared to their baseline values and when compared to those only taking the DPP-4 inhibitor, and

5. experience decreased appetite before a standardized meal, increased satiety during that meal when compared to their baseline values and when compared to those only taking the DPP-4 inhibitor, and

6. are found to have elevated GLP-1 as well as PYY levels with reduced ghrelin levels at 1 hour after the both the OGTT and the standardized meal when the values are compared to their baseline values and when compared to those only taking the DPP-4 inhibitor.

### General

In this study, T2D patients are randomly assigned to either consume 180 ml of Activated Soy Pod Fiber or a placebo formula containing cellulose orally within 1 hour prior to either meals 1 or 2 as well as within 1 hour prior to meal 3 each day. Patients and experimenters are blinded to this assignment. All patients are also instructed to take sitagliptin (Januvia®) at the recommended dose of 100 mg, once per day in the morning prior to meal 1 as a treatment to manage their diabetes.

Subjects report weekly for measurements and assessment of any side effects. They are asked to collect a stool sample before the initiation of the trial as well as at the end of the 4 week treatment period. They are also asked to record any side effects and their frequency (checklist assessment). They are asked to record appetite (how hungry are you) and satiety (how full are you) during the OGTT at both the onset and at the end of the trial as well as before and during a standardized meal at the 3^{rd} week of treatments. They are provided with the proper paper work to record these.

### Subject Screening and Selection

A total of 24 subjects is selected. 12 will be randomly assigned to receive sitagliptin+placebo (a cellulose solution that contains the same total dietary fiber content as Activated Soy Pod Fiber and mimics Activated Soy Pod Fiber in color and taste) or sitagliptin+Activated Soy Pod Fiber.

### Qualifications of Subjects

1) T2D men and women between the ages of 18 and 70 with a BMI between 25 and 45 are eligible.

2) Fasting blood glucose between greater than 125 mg/dl.

3) Stable weight over 2 months

Subjects Excluded from the Study

### People who:

a) take medications affecting glucose other than sitagliptin,

b) take medications affecting insulin other than sitagliptin,

c) take medications affecting body weight,

d) take medications affecting bacterial flora,

e) have intestinal disease or a recent history of intestinal disease,

f) have had surgery on stomach or intestine,

g) are hypothyroid, h) are pregnant,

i) have heart disease.

### Laboratory Evaluation

Different tests are performed at the screening of potential participants, at the beginning of the study, and at the end of the 4 week treatment period.

4) Screening: Subjects are screened to exclude hypothyroidism, pregnancy, and heart disease. The following tests can suffice for this: T4, T3, TSH, urine pregnancy test, blood pressure & ECG. Fasting blood glucose, fasting insulin and HgbA1 levels are also measured as an assessment of their diabetic state.

5) Beginning of study: Subjects passing the initial screen are evaluated at the beginning of

Week #1 as follows:

a) Fasting blood glucose, insulin, and HgbA1 levels.

b) SMA 20, including uric acid and liver function tests

c) Blood Triglycerides

d) Plasma Cholesterol, including fractions

f) Weight, taken on the same scale each time

g) Body fat % and total body fat, determined by DXA.

h) Height

i) Waist and hip measurements

j) Blood glucose, insulin, GLP-I, PYY and ghrelin responses to a 75 g oral glucose challenge

k) Assessment of appetite and satiety before, during and after a standardized meal using a visual analog scale

l) Stool is collected into a preservative and analyzed within 1 week at baseline and at the end of study.

6) End of study assessment:

c) all labs and assessments done in step 2 at beginning of study,

d) Analysis of the fecal microbiome DNA from both the initial sample and the final sample.

e) stool pH:

f) stool SCFA.

g) stool lactoferrin

h) stool white blood cells

i) stool mucus

j) stool secreted immunoglobulin A

k) stool anti-gliadin secreted immunoglobulin A

l) stool pathogenic bacteria

m) stool yeast, fungi, and parasites

n) stool triglycerides

o) stool branched chain fatty acids

p) stool long chain fatty acids

q) stool cholesterol.

Patients selected for participation are allowed an ad libitum diet and are given an evaluation sheet to assess their appetite and satiety. Foods excluded include alcohol. Low calorie liquids are stressed in place of high calorie liquids such as fruit juices, milk, sweet tea (tea with sugar), regular soft drinks, coffee with sugar, etc. All 24 patients are also instructed to take sitagliptin (Januvia®) at the recommended dose of 100 mg, once per day in the morning with or without food as a treatment to manage their diabetes. 12 patients are randomly selected to also consume Activated Soy Pod Fiber before 2 of 3 daily meals and the remaining 12 patients are instructed to consume a placebo before 2 of 3 daily meals. Patients and investigators are blinded to whether the snack replacement is placebo or Activated Soy Pod Fiber.

### Duration

Subjects are given a 4 weeks supply of sitagliptin and either Activated Soy Pod Fiber or placebo at the onset and are instructed to consume the entire 180 ml volume of either snack replacement within 1 hour prior to either meals 1 or 2, as well as another 180 ml volume of snack replacement within 1 hour prior to meal 3. All subjects are required to take 1 tablet of sitagliptin daily (100 mg) in the morning with or without food. Ad libitum diets are followed for 4 weeks.

### Outcome

This study is designed to exemplify that Activated Soy Pod Fiber:

1) Shifts bacterial species in fecal samples from those documented in feces from T2D to those typical of non-diabetic subjects when samples at the end of study from those assigned to Activated Soy Pod Fiber are compared to samples at the onset of study and when subjects taking sitagliptin+Activated Soy Pod Fiber are compared to patients taking sitagliptin+placebo.

2) Improves the blood glucose and insulin responses to an OGTT by decreasing the areas under the insulin curve (improved insulin sensitivity) when subjects taking sitagliptin+Activated Soy Pod Fiber are compared to patients taking sitagliptin+placebo

3) produces weight loss, loss of body fat, and (or) decrease of body fat % when patients assigned to Activated Soy Pod Fiber are compared to samples at the onset of study and when subjects taking sitagliptin+Activated Soy Pod Fiber are compared to patients taking sitagliptin+placebo.

4) produces decreased fasting blood glucose levels when subjects taking sitagliptin+Activated Soy Pod Fiber are compared to patients taking sitagliptin+placebo, and

5) Increases GLP-1 and PYY response to the oral glucose challenge and decreases the fasting ghrelin levels prior to the OGTT when patients assigned to Activated Soy Pod Fiber are compared to samples at the onset of study and when subjects taking sitagliptin+Activated Soy Pod Fiber are compared to patients taking Sitagliptin+placebo,

6) Patients assigned to the Activated Soy Pod Fiber arm when compared to samples at the onset of study and subjects taking Sitagliptin Activated Soy Pod Fiber when compared to patients taking Sitagliptin+placebo are expected to demonstrate the following changes in stool samples:

a) Decreases stool pH;

b) Increases stool SCFA.

c) Increased stool lactoferrin

d) Decreased stool white blood cells

e) Decreased stool mucus

f) Increased stool secreted immunoglobulin A

g) Increased stool anti-gliadin secreted immunoglobulin A

h) Decreased stool pathogenic bacteria

i) Decreased stool yeast, fungi, and parasites

j) Increased stool triglycerides

k) Decreased stool branched chain fatty acids

l) No change in stool long chain fatty acids

m) Increased stool cholesterol.

If subjects took Activated Soy Pod Fiber with other DPP-IV inhibitors or other formulations of sitagliptin, the subjects are expected to also have significantly improved glucose regulation as measured by reduced HgbA1c levels.

### Example 7

### Study Utilizing Activated Soy Pod Fiber Snack Replacement to Correct the GI Dysbiosis Observed in Overweight and Obese Children, and Improve Glucose Regulation and Improve Body Composition

### Subjects and Methods

The required number of children are properly screened to fulfill the necessary qualifications and their parental consent is obtained, appropriate laboratory evaluations are performed, measures of positive primary and secondary outcome responses are recorded, adverse events are documented, and children and their parents are adequately followed-up.

### Overview

This study is designed to exemplify that overweight children with prediabetes or at high risk of developing T2D (type 2 diabetes) on an ad libitum diet who take Formula A (identical active ingredients to Activated Soy Pod Fiber but formulated in a child friendly delivery system such as ice cream, jelled animals, cookies, etc.) within 1 hour prior to either meal 1 or meal 2, as well as within 1 hour prior to meal 3 for 4 weeks:

1. Eliminate stool characterized as normal diversity when compared to the start of the intervention, and

2. Have an improved oral glucose tolerance test (OGTT) as measured by blood glucose and insulin levels before, during, and at 120 minutes after ingestion of 1.75 g glucose/kg body weight up to 75 g glucose when compared to their initial OGTT, and

3. experience an improved body composition as measured by a decrease in body weight, a decrease in body fat or % body fat, a decrease in waist circumference measurements, and

4. Experience decreased fasting blood glucose levels

5. experience decreased appetite before a meal, increased satiety during the meal, and

6. are found to have elevated GLP-I as well as PYY levels with reduced ghrelin levels 1 hour after the OGTT when the values are compared to those at the initiation of the trial.

### General

In this study, children consume 6 jelled animals of Formula B formula (each jelled animal contains about 20 g of Formula B) within 1 hour prior to either meals 1 or 2 as well as within 1 hour prior to meal 3 each day.

Subjects report weekly for measurements and assessment of any side effects. They are asked to collect a stool sample before initiating Formula B intervention as well as at the end of the 4 week treatment period. They are also asked to report side effects to their parents who record them and their frequency (checklist assessment). The parents are instructed to ask and to record appetite (how hungry are you) and satiety (how full are you) before, during, and after a standardized 3^{rd} meal at the beginning of study and at the end. The investigators score the same assessment during the OGTT at both the onset and at the end of the trial as well as at home. The parents are provided with the proper paper work to record these.

### Subject Screening and Selection

A total of 10 children is selected.

### Qualifications of Subjects

1) Healthy prepubertal boys and girls between the ages of 7 and 12 with a BMI between 25 and 30 are eligible.

2) Fasting blood glucose between 100 and 125 mg/dl.

Subjects Excluded from the Study

### Children who:

a) take medications affecting glucose,

b) take medications affecting insulin,

c) take medications affecting body weight,

d) take medications affecting bacterial flora,

e) have intestinal disease or a recent history of intestinal disease,

f) have had surgery on stomach or intestine,

g) are hypothyroid.

### Laboratory Evaluation

Different tests are performed at the screening of potential participants, at the beginning of the study, and at the end of the 4 week treatment period.

### Screening

Children are screened to exclude hypothyroidism and puberty. The following tests can suffice for this: T4, T3, TSH, a physical exam, and in questionable cases based on the physical exam or peripubertal presentations, a gonadotropin-releasing hormone challenge test.

### Beginning of Study

Children passing the initial screen are evaluated at the beginning of WEEK #1 as follows:

a) Fasting blood glucose and insulin levels.

b) SMA 20, including uric acid and liver function tests

c) Triglycerides

d) Cholesterol, including fractions

e) Glycosylated hemoglobin A1 (HgbA1)

f) Weight, taken on the same scale each time

g) Body fat % and total body fat, determined by DXA.

h) Height

i) Waist and hip measurements

j) Blood glucose, insulin, GLP-I, PYY and ghrelin responses to a 1.75 g/kg (up to 75 g) oral glucose challenge

k) Assessment of appetite and satiety using a visual analog scale

(l) Stool is collected in a preservative and analyzed within 1 week.

### End of Study Assessment

a) all labs and assessments done in step 2 at beginning of study,

b) Analysis of the fecal microbiome DNA from both the initial sample and the final sample.

### Study Design

Children selected for participation are allowed an ad libitum diet and their parents are given an evaluation sheet to assess their appetite and satiety. Low calorie liquids are stressed in place of high calorie liquids such as fruit juices, milk, regular soft drinks, coffee with sugar, etc. The children are encouraged to consume Formula B as their only between meal snack. Other snacks such as candy, ice cream, milk shakes, cookies, potato chips, etc. are discouraged.

### Duration

Children are given a 4 weeks supply of Formula B at the onset and are instructed to consume the entire 6 jelled animals containing Formula B within 1 hour prior to either meal 1 or 2, as well as another 6 jelled animals containing Formula B within 1 hour prior to meal 3. Ad libitum diets are followed for 4 weeks, but the children are instructed to consume Formula B as their only between meal snack.

### Outcome

This study is designed to exemplify that Formula B:

1) Shifts bacterial species in fecal samples from those documented for obese children to those typical of healthy lean children and adolescents when samples at the end of study are compared to samples at the onset of study.

2) Improves the blood glucose and insulin responses to an OGTT by decreasing the areas under the insulin curve (improved insulin sensitivity)

3) produces weight loss, loss of body fat, and (or) decrease of body fat %

4) Increases GLP-1 and PYY response to the oral glucose challenge and decreases the fasting ghrelin levels prior to the OGTT when comparing final values to the initial measurements.

5) Alters the following stool characteristics:

a) Decreases stool pH;

b) Increases stool SCFA.

c) Increased stool lactoferrin

d) Decreased stool white blood cells

e) Decreased stool mucus

f) Increased stool secreted immunoglobulin A

g) Increased stool anti-gliadin secreted immunoglobulin A

h) Decreased stool pathogenic bacteria

i) Decreased stool yeast, fungi, and parasites

j) Increased stool triglycerides

k) Decreased stool branched chain fatty acids

l) No change in stool long chain fatty acids

m) Increased stool cholesterol.

If children took Activated Soy Pod Fiber for periods longer than 4 weeks as a snack replacement, particularly for at least 8 weeks, the children would experience significant weight loss that was primarily fat loss.

### Example 8

### Human Study Utilizing Either Activated Soy Pod Fiber or a Placebo to Shift the Gastrointestinal Microbiota in Inflammatory Bowel Disease (IBD) to that Characterized in Healthy Individuals

### Subjects and Methods

The required number of subjects are properly screened to fulfill the necessary qualifications, appropriate laboratory evaluations are performed, measures of positive primary and secondary outcome responses are recorded, adverse events are documented, and patients are adequately followed-up.

### Overview

This randomized, placebo-controlled clinical trial is designed to exemplify the efficacy and tolerability of Activated Soy Pod Fiber in diarrhea-predominant humans with IBD. Subjects assigned to consume Activated Soy Pod Fiber but not a placebo, within 1 hour prior to consuming either meal 1 or meal 2, as well as within 1 hour prior to consuming meal 3 for 4 weeks:

1. Eliminate firm stool containing a normal diversity of GI microbiota within 1 week of starting treatment

2. Report adequate relief for all 4 weeks, and

3. Report decreased urgency, and

4. Report decreased stool frequency, and

5. Report relief of abdominal pain

### General

In this study, subjects are randomly selected to consume 180 ml of either Activated Soy Pod Fiber formula or placebo formula orally within 1 hour prior to ingestion of either meals 1 or 2 as well as within 1 hour prior to consumption of meal 3 each day. The subjects and experimenters are blinded to the treatment assignments. Placebo formula contains cellulose with food coloring and flavor to match the total dietary fiber content of Activated Soy Pod Fiber. Placebo is prepared by Merlin Development at the same time they prepare Activated Soy Pod Fiber. Both formulations are coded by Merlin Development and the code is maintained with them as well as is held in confidence by a pharmacist at the study clinic until all data are collected at the end of study.

Subjects report weekly for measurements and assessment of any side effects. They are asked to collect a stool sample before initiating Activated Soy Pod Fiber or Placebo intervention as well as at the end of the 4-week treatment period. During the screening, treatment, and follow-up periods, daily and weekly symptom data are collected using an interactive telephone-based system.

Pain and bowel function data are collected during the screening phase to ensure that patients had a suitable symptom level at study entry. Severity of pain and discomfort was assessed daily on a 5-point scale (0, none; 1, mild; 2, moderate; 3, intense; and 4, severe). Stool consistency data are monitored daily and scored as follows: 1, very hard; 2, hard; 3, formed; 4, loose; and 5, watery. Absence of stool was assigned a value of 0. Patients also record their IBD symptoms urgency (0%, feel no need to evacuate-100%, feel severe need to evacuate), stool frequency (# of stools per day), bloating (0, no sensation of extended abdomen; 1, mild; 2, moderate; 3, severe) and sense of incomplete evacuation (0, sensation of complete evacuation; 1, incomplete; 2, constipated) daily during the treatment and follow-up phases.

### Subject Screening and Selection

Patients with IBD and a diarrhea-predominant bowel pattern aged 18 years or older are enrolled in this study if their symptoms fulfilled the Rome I criteria for IBD for at least 6 months. Patients undergo a 2-week screening evaluation to confirm sufficient level of pain and stool consistency before randomization.

Patients are excluded if they are pregnant breastfeeding, or not using approved methods of contraception (if of child-bearing potential); if an unstable medical or other gastrointestinal condition exists; if there is a major psychiatric disorder or substance abuse within the previous 2 years; if an investigational drug was used within 30 days of the screening phase; or if a prohibited concurrent medication (likely to interfere with gastrointestinal tract function or analgesia) was used within 7 days before entering the screening phase. Pain and bowel function data are collected during the screening phase to ensure that patients had a suitable symptom level at study entry as described above.

### Symptom and Laboratory Evaluation

Evaluations are performed at the screening of potential participants, at the beginning of the study, daily, and at the end of the 4-week treatment period.

1) Beginning of study and daily

a) Severity of pain and discomfort is assessed on a 5-point scale (0, none; 1, mild; 2, moderate; 3, intense; and 4, severe).

b) Stool consistency data are scored as follows: 1, very hard; 2, hard; 3, formed; 4, loose; and 5, watery. Absence of stool was assigned a value of 0.

c) Urgency (0%, feel no need to evacuate-100%, feel severe need to evacuate),

d) Stool frequency (# of stools per day)

e) Bloating (0, no sensation of extended abdomen; 1, mild; 2, moderate; 3, severe)

f) Sense of incomplete evacuation (0, sensation of complete evacuation; 1, incomplete; 2, constipated)

g) Body weight.

2) Beginning of study

Stool is collected and stored frozen but not analyzed until the end of study.

3) End of study assessment

Analysis of the fecal microbiome DNA and complete stool analysis from both the initial sample and the final sample.

### Study Design

Subjects selected for participation are allowed an ad libitum diet. Foods excluded include alcohol. The subjects are encouraged to consume either Activated Soy Pod Fiber or Placebo within 1 hour prior to 2 meals each day with ingestion of the test agent being mandatory prior to the 3^{rd} meal.

Subjects are given a 4 week supply of ether Activated Soy Pod Fiber or Placebo at the onset and are instructed to consume the entire 180 ml volume containing either formula within 1 hour prior to either meals 1 and 2, as well as another 180 ml volume containing either formula within 1 hour prior to meal 3.

Subjects report weekly for measurements and assessment of IBSD symptoms.

During the screening and treatment periods, daily symptom data are collected using an interactive telephone-based system.

### Outcome

This study is designed to exemplify that Activated Soy Pod Fiber and not Placebo:

1) Improves severity of pain and discomfort;

2) Increases stool consistency;

3) Decreases urgency to evacuate,

4) Decreases stool frequency;

5) Decreases bloating

6) Increases sense of complete evacuation.

7) Shifts stool profile to one representative of a healthy GI microbiome

a) Decreases stool pH:

b) Increases stool SCFA.

c) Increased stool lactoferrin

d) Decreased stool white blood cells

e) Decreased stool mucus

f) Increased stool secreted immunoglobulin A

g) Increased stool anti-gliadin secreted immunoglobulin A

h) Decreased stool pathogenic bacteria

i) Decreased stool yeast, fungi, and parasites

j) Increased stool triglycerides

k) Decreased stool branched chain fatty acids

l) No change in stool long chain fatty acids

m) Increased stool cholesterol.

Utilization of Activated Soy Pod Fiber to treat idiopathic diarrhea such as a parasitic infection, a viral infection and a symptomatic response to a food is expected to also improve the severity of pain and discomfort, increase stool consistency, decrease the urgency to evacuate, decrease stool frequency, and decrease the sensation of bloating.

### Example 9

### Human Study Utilizing Either Activated Soy Pod Fiber or a Placebo to Treat Gestational Diabetes

### Subjects and Methods

The required number of subjects are properly screened to fulfill the necessary qualifications, appropriate laboratory evaluations are performed, measures of positive primary and secondary outcome responses are recorded, adverse events are documented, and patients are adequately followed-up.

### Overview

This randomized, placebo-controlled clinical trial is designed to exemplify the efficacy and tolerability of Activated Soy Pod Fiber in gestational diabetes. Subjects assigned to consume Activated Soy Pod Fiber but not a placebo, within 1 hour prior to consuming either meal 1 or meal 2, as well as within 1 hour prior to consuming meal 3 for 4 weeks:

1. Have improved glycemic control

2. Have acceptability of the treatment

### General

In this study, subjects are randomly selected to consume 180 ml of either Activated Soy Pod Fiber formula or placebo formula orally within 1 hour prior to ingestion of either meals 1 or 2 as well as within 1 hour prior to consumption of meal 3 each day. The subjects and experimenters are blinded to the treatment assignments. Placebo formula contains cellulose with food coloring and flavor to match the total dietary fiber content of Activated Soy Pod Fiber. Placebo is prepared by Merlin Development at the same time they prepare Activated Soy Pod Fiber. Both formulations are coded by Merlin Development and the code is maintained with them as well as is held in confidence by a pharmacist at the study clinic until all data are collected at the end of study.

Subjects report weekly for measurements and assessment of any side effects. They are asked to collect a stool sample before initiating Activated Soy Pod Fiber or Placebo intervention as well as at the end of the 4 week treatment period. During the screening, treatment, and follow-up periods, daily blood glucose data are collected by the patient using finger stick.

### Subject Screening and Selection

Women who are at least 18 years of age with gestational diabetes at 24-28 weeks (American Diabetes Association (ADA) criteria), who need an intervention treatment following the failure of the diet and exercise, and who are not known type 1 or type 2 diabetics, who are not being treated with a medicine that interferes with glucose metabolism, who have no allergies to soy, do not have preeclampsia, are not taking antibiotics, and are not taking proton pump inhibitors.

### Symptom and Laboratory Evaluation

Glycemic control is evaluated during treatment and 8-12 weeks following delivery. Stool analysis before treatment is initiated, during treatment and after 8-12 weeks after delivery.

### Study Design

Women selected for participation are diagnosed with gestational diabetes between 24 and 28 weeks of pregnancy that is resolved after 10 days of diet and exercise. The subjects are encouraged to consume either Activated Soy Pod Fiber or Placebo within 1 hour prior to 2 meals each day with ingestion of the test agent being mandatory prior to the 3^{rd} meal in addition to dietary advice.

Women are given an 8 week supply of ether Activated Soy Pod Fiber or Placebo at the onset and are instructed to consume the entire 180 ml volume containing either formula within 1 hour prior to either meals 1 and 2, as well as another 180 ml volume containing either formula within 1 hour prior to meal 3.

Subjects measure fasting blood glucose each morning by finger stick and report the values weekly during office visits. Comparison of the treatment group to the placebo group are made from 2-3 weeks of treatment and at 8-12 weeks following delivery.

### Outcome

This study is designed to exemplify that Activated Soy Pod Fiber and not Placebo:

1) Improves glucose regulation until term;

2) Has no serious adverse events:

3) Does not alter rates of caesarian section, preterm delivery, neonatal mortality, number of neonatal and maternal trauma related to delivery, number of days of hospitalization

2) Shifts stool profile to one representative of a healthy GI microbiome

n) Decreases stool pH;

o) Increases stool SCFA.

p) Increased stool lactoferrin

q) Decreased stool white blood cells

r) Decreased stool mucus

s) Increased stool secreted immunoglobulin A

t) Increased stool anti-gliadin secreted immunoglobulin A

u) Decreased stool pathogenic bacteria

v) Decreased stool yeast, fungi, and parasites

w) Increased stool triglycerides

x) Decreased stool branched chain fatty acids

y) No change in stool long chain fatty acids

z) Increased stool cholesterol.

### Example 10

### Effects of Activated Soy Pod Fiber on the Progression of Type 1 Diabetes in New Onset Subjects

### Subjects and Methods

The required number of subjects are properly screened to fulfill the necessary qualifications, appropriate laboratory evaluations are performed, measures of positive primary and secondary outcome responses are recorded, adverse events are documented, and patients are adequately followed-up.

### Overview

This randomized, placebo-controlled clinical trial is designed to exemplify the efficacy and tolerability of Activated Soy Pod Fiber in newly diagnosed T1D. Subjects assigned to consume Activated Soy Pod Fiber but not a placebo, within 1 hour prior to consuming a meal for 3 months:

1. Have improved glycemic control

2. Have preserved residual insulin secretion

3. Have maintained beta cell function

4. Have acceptability of the treatment

### General

In this study, subjects are randomly selected to consume 180 ml of either Activated Soy Pod Fiber formula or placebo formula orally within 1 hour prior to ingestion of all meals. The subjects and experimenters are blinded to the treatment assignments. Placebo formula contains food coloring and flavor to match the total dietary fiber content of Activated Soy Pod Fiber. Placebo is prepared by Merlin Development at the same time they prepare Activated Soy Pod Fiber. Both formulations are coded by Merlin Development and the code is maintained with them as well as is held in confidence by a pharmacist at the study clinic until all data are collected at the end of study.

Subjects report monthly for measurements and assessment of any side effects. They are asked to collect a stool sample before initiating Activated Soy Pod Fiber or Placebo intervention as well as at monthly clinic visits. During the screening, treatment, and follow-up periods; the patient using finger stick collects daily blood glucose data.

The study is a randomized, two-arm, trial in which ⅔ of participants will receive the Activated Soy Pod Fiber, while the remaining ⅓ will receive a placebo. Participants will need to return to the clinical center for a visit every month for 3 months; those participants that continue to secrete insulin will have further follow-up for an additional year.

### Subject Screening and Selection

Inclusion Criteria:
Between the ages of 8 and 45 years
Within 3 months of diagnosis of type 1 diabetes
Have presence of at least one diabetes-related autoantibody
Must have stimulated C-peptide levels of at least 0.2 pmol/ml measured during a mixed meal tolerance test (MMTT) during screening
If female with reproductive potential, willing to avoid pregnancy and undergo pregnancy testing while participating in the study
Have not received an immunization for at least one month
Must be willing to comply with intensive diabetes management
Must weigh at least 25 kg at study entry

### Exclusion

### Criteria:

Are immunodeficient or have clinically significant chronic lymphopenia
Have an active infection or positive purified protein derivative (PPD) test result
Currently pregnant or lactating; or anticipate becoming pregnant.
Require chronic use of steroids
Have current or past HIV, hepatitis B, or hepatitis C infection
Have any complicating medical issues that interfere with study conduct or cause increased risk
Have a history of malignancies
Currently using non-insulin pharmaceuticals that effect glycemic control
Currently participating in another type 1 diabetes treatment study

### Primary Outcome Measures:

Area under the stimulated C-peptide curve over the first 2 hours of a 4-hour mixed meal tolerance test (MMTT) administered at the end of 3 months is expected to be increased when compared to placebo and maintained from that observed at time of screening.

### Secondary Outcome Measures:

Insulin dose (units/kg) and number of injections are expected to be maintained from screening and decreased when compared to placebo
Glycosylated hemoglobin (HbA1c) is expected to be maintained from screening and decreased when compared to placebo
Blood cytokine levels are expected to be maintained from screening and decreased when compared to placebo
Stool pH is expected to be decreased in the Activated Soy Pod Fiber group
Stool SCFAs are expected to be increased in the Activated Soy Pod Fiber group.
Stool immunoglobulin A is expected to be increased in the Activated Soy Pod Fiber group.

### Example 11

### Production of Activated Soy Pods

Thirty (30) soy plants of a variety used for edamame (IA2032) were planted and grown in a greenhouse. Twenty (20) pods were harvested from 3 plants at the reproductive stage 6 (R6) or when the green pod contains seeds (beans) that have filled the pods. The pods were placed in a plastic bag and carried to a laboratory. Five (5) pods were transferred into a 50 ml centrifuge tube that was filled with Millipore water. A top was placed on the tube and the tube was inverted several times to wash the pods. The water was drained and the rinse was repeated. Three other sets of 5 pods were washed in a similar manner.

All 20 pods were thinly sliced by placing pod containing seeds vertically in the food pusher of a food processor that is modified with a 20 ml syringe in the center. The pod is placed into the syringe, which holds the pod vertical and delivers it to the slicing blade about 2 mm from the cutting surface. The food processor (KitchenAid®, Model KFP720WH1) using disc-slicing attachment was turned on slicing the pods and seeds into thin cross sections. The sliced pod tissue was transferred into a four 150 mm Petri dishes containing S & S Blue Ribbon #589 filter paper that is presoaked with 6 ml of Millipore water.

Tissue from 1 Petri dish was immediately transferred to a 50 ml centrifuge tube and placed in a freezer at −80 C. These samples represent 0 h of incubation. The other Petri dishes containing sliced pods were placed into a sealed desiccator on a platform to isolate them from a dish of saturated potassium chloride below the platform to fix the humidity at 83%. The desiccator was placed in the dark.

After incubation for 24 h, 48 h or 72 h at 22.5 C the tissue was transferred into a 50 ml centrifuge tube and then stored at −80 C. The centrifuge lids were removed and the tubes were covered with Chem Wipe tissue that was held in place by a rubber band. The tubes were placed into a freeze dryer overnight.

Dried tissue was milled using a Glen Mills mill with a 1.0 mm screen. The dried tissue was transferred into hopper of the mill and powder was collected. The mill screen was changed to 0.5 mm size and the previously milled material was added to the hopper and milled to produce a fine powder.

In order to measure soy compounds activated by this process high performance liquid chromatography (HPLC) was performed. 0.2 g powder was weighed into a 1 ml microfuge tube. 1 ml methanol was added and the tubes were sonicated in a water bath with water level adjusted just so the vials float. After 1 h of sonication, the microfuge tubes were centrifuged at high speed for 5 min. The supernatant was transferred onto the filter of a 1 ml centrifuge filter and centrifuged for 5 min to remove fine particles. That filtrate was transferred to an autosampler vial for HPLC analysis (HPLC analyses were performed on a Waters 2695 combined with a Waters UV-visible 2996 photodiode array detector (Waters Associated, Milford, Mass.).

Soy compounds were separated using a Luna II C18 reverse phase column (4.6×250 mm; 5 μm; Phenomenex, Torrance, Calif.). A guard column containing the same packing was used to protect the analytical column. Solvent A was 0.1% acetic acid in water and solvent B was 100% acetonitrile.

A 20 μl volume of sample was injected and the HPLC was programmed with a flow rate of 1.0 ml/min using 15% B for 8 min, then 58% B in 50 min, then 90% B in 10 min followed by holding at 90% B for 10 min. The spectra was collected between 220 and 400 nm by a photodiode array detector.

### Data

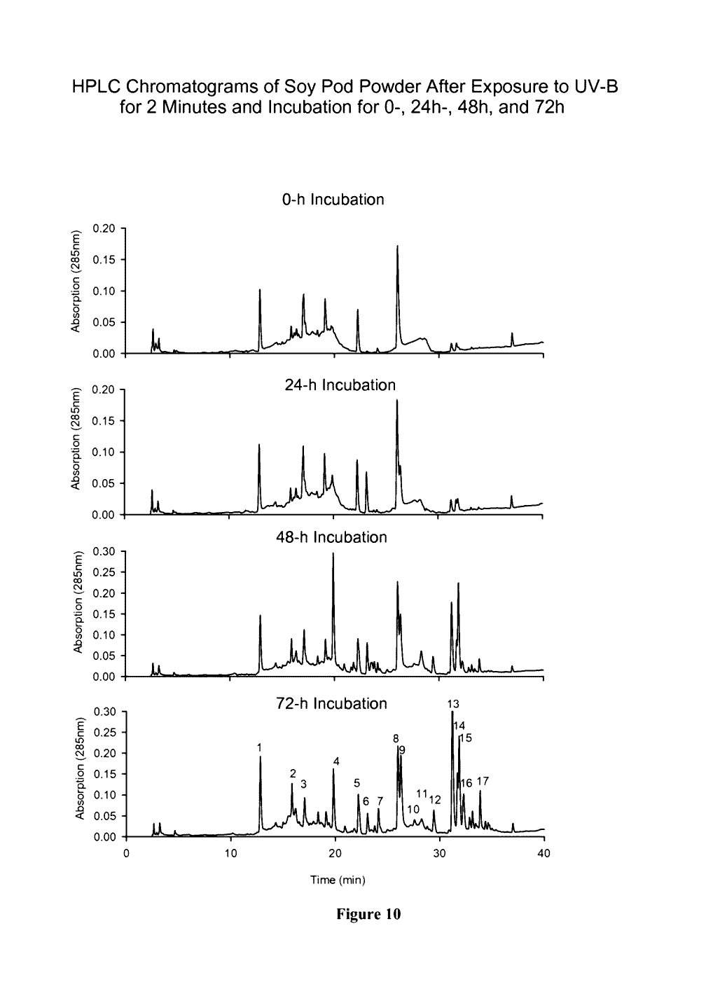

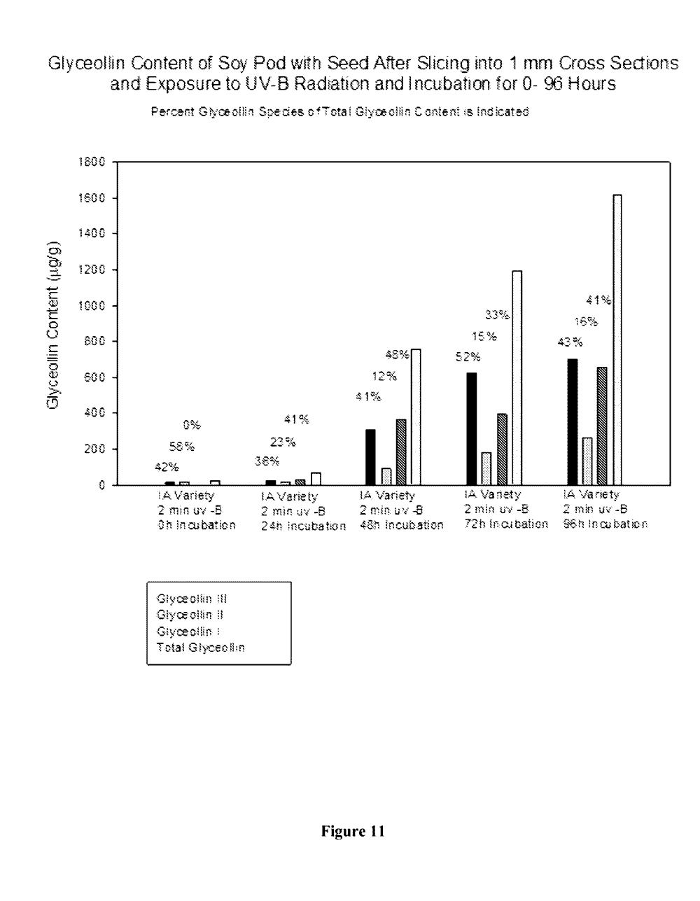

FIG. 10 is a HPLC profile demonstrating species of soy compounds observed without incubation (0 h incubation) and new peaks of UV absorption with incubation for up to 72 h. Incubation is required for the enzyme systems in the plant tissue to process new molecules in response to physical injury (slicing). In particular, peaks identified at 72 h represent glyceollin III (peak 13), glyceollin II (peak 14) and glyceollin I (peak 15). Also identified is coumestrol (peak 9) and glycinol (peak 4). The unknown peaks are being identified.

It is clear from these data that such processing is a useful means of activating soy pod tissue to produce bioactive molecules.

FIG. 11 shows quantification of 3 glyceollin species produced by slicing and exposure to uv radiation. One bar represents summation of the 3 glyceollin species that is as high as 1.5 mg/g powder if the incubation is performed for 96 h. The most abundant species are glyceollin 111 and glyceollin I, which together represent about 80% of glyceollin generated.

### Example 12

### Large Scale Production of Activated Soy Pods

Eight hundred (800) soy plants of a variety used for oil (Pioneer 95Y61) were planted and grown on the LSU AgCenter Dean Lee Research and Extension Center, Alexandria, La. Three hundred twenty thousand (320,000) pods were harvested at the reproductive stage 6 (R6) or when the green pod contains seeds (beans) that have filled the pods. The pods were placed into plastic bags that were sealed, placed on ice, and transported to a laboratory. The bags were placed at 4° C. overnight. The next morning each bag of soy pods were washed 3-times with distilled water.

All pods containing seeds were thinly sliced by placing pod containing seeds vertically in the food pusher of a food processor that is modified with a 20 ml syringe in the center. The pod is placed into the syringe, which holds the pod vertical and delivers it to the slicing blade about 2 mm from the cutting surface. The food processor (KitchenAid®, Model KFP720WH1) using disc-slicing attachment was turned on slicing the pods and seeds into thin cross sections. The sliced pod tissue was transferred into cafeteria trays (9 in×12 in) containing a paper towel that is presoaked with distilled water.

Tissue from about 30,000 pods was immediately transferred to gallon size plastic bags that were sealed and placed in a freezer at −80 C. These samples represent unactivated soy pods. The other sliced pods that were placed on trays were placed into Rubbermaid Roughneck containers (10 in height×11 in wide×11 in deep) using Petri dishes as spacers between each tray. Two 150×15 mm Petri dishes were filled with saturated potassium chloride and placed below the bottom cafeteria tray to fix the humidity at 83% after the container was sealed. Ten (10) containers were placed in the dark for 72 h.

After incubation at 22.5 C the tissue from each container was transferred into gallon-sized plastic bags and then stored at −80 C. Each bag of activated (incubated for 72 h at 22.5 C) and unactivated tissue was placed into a freeze dryer for 4 days to remove the water.

Dried tissue was milled using a Retsch Cutting Mill SM 100 with a 0.5 mm screen. The dried tissue was transferred into hopper of the mill and powder was collected. Milled material from boxes 1 and 2 as well as boxes 5 and 7 were pooled (see Table 2 below).

In order to measure soy compounds activated by this process high performance liquid chromatography (HPLC) was performed. 0.2 g powder from each pool of milled soy pod fiber was weighed into a 1 ml microfuge tube. 1 ml methanol was added and the tubes were sonicated in a water bath with water level adjusted just so the vials float. After 1 h of sonication, the microfuge tubes were centrifuged at high speed for 5 min. The supernatant was transferred onto the filter of a 1 ml centrifuge filter and centrifuged for 5 min to remove fine particles. That filtrate was transferred to an autosampler vial for HPLC analysis (HPLC analyses were performed on a Waters 2695 combined with a Waters UV-visible 2996 photodiode array detector (Waters Associated, Milford, Mass.).

Soy compounds were separated using a Luna II C18 reverse phase column (4.6×250 mm; 5 μm; Phenomenex, Torrance, Calif.). A guard column containing the same packing was used to protect the analytical column. Solvent A was 0.1% acetic acid in water and solvent B was 100% acetonitrile.

A 20 μl volume of sample was injected and the HPLC was programmed with a flow rate of 1.0 ml/min using 15% B for 8 min, then 58% B in 50 min, then 90% B in 10 min followed by holding at 90% B for 10 min. The spectra was collected between 220 and 400 nm by a photodiode array detector.

### Data

Glyceollin content in milled soy pod fiber from each box or from pooled boxes is shown in the Table 2 below. The greatest production of glyceollin was from material incubated in boxes 5 and 7. There was no glyceollin produced in boxes 8 or 10. Sliced soy pods that were immediately frozen and not incubated served to control for the glyceollin biosynthesis and did not produce any of the glyceollins (unactivated soy pod fiber).

TABLE 2
Glyceollin content of milled activated-
and unactivated- (Con) soy pod fiber.

| | Gly I | Gly II | Gly III | Total Gly | Total Mass |
|---|---|---|---|---|---|
| Box # | (μg/g) | (μg/g) | (μg/g) | (μg/g) | (g) |
| 1 & 2 | 103.89 | 33.08 | 19.72 | 156.69 | 359.3 |
| 3 | 107.50 | 37.91 | 28.99 | 174.40 | 135.60 |
| 4 | 43.45 | 11.55 | 7.76 | 62.75 | 183.1 |
| 5 & 7 | 736.91 | 204.39 | 225.13 | 1166.43 | 548.0 |
| 6 | 110.9 | 31.56 | 25.04 | 167.50 | 347.7 |
| 8 | 0 | 0 | 0 | 0 | 236.0 |
| 9 | 148.34 | 50.72 | 27.43 | 226.50 | 186.4 |
| 10 | 0 | 0 | 0 | 0 | 229.2 |
| Con | 0 | 0 | 0 | 0 | 907.9 |

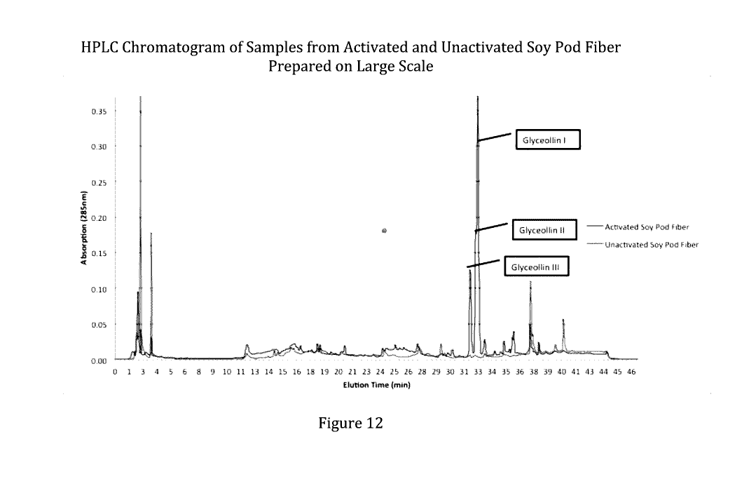

FIG. 12 is a HPLC profile demonstrating species of soy compounds observed in the unactivated soy pod fiber (Red Curve) and new peaks of UV absorption with incubation for 72 h (Activated Soy Pod fiber (Blue Curve)). Incubation is required for the enzyme systems in the plant tissue to process new molecules in response to physical injury (slicing). In particular, peaks identified in only the activated soy pod fiber were glyceollin III, glyceollin II, and glyceollin I. Several other peaks were identified in the activated soy pod fiber and are being identified. They are believed to other isoflavones that are only produced by tissue injury such as slicing. It is clear from these data that such processing is a useful means of activating soy pod tissue to produce novel bioactive molecules.

The macronutrient content for activated soy pod fiber is similar to that of the unactivated soy pod fiber. Both contain soy protein and sol oil (fat). There is also a significant amount of novel total dietary fiber (Table 3).

TABLE 3
Macronutrient content of milled activated-
and unactivated- (Con) soy pod fiber

| | Activated Soy | Unactivated Soy |
|---|---|---|
| Macronutrient | Pod Fiber | Pod Fiber |
| Total Carbohydrate | 47.9% | 47.5% |
| Available Carbohydrate | 13.3% | 20.0% |
| Protein | 30.0% | 28.8% |
| Fat | 13.4% | 16.1% |
| Total Dietary Fiber | 38.7% | 31.3% |
| Soluble Fiber | 4.1% | 3.8% |
| Insoluble Fiber | 34.6% | 27.5% |
| Calories (Insol fiber subtracted) | 294 Cal/100 g | 340 Cal/100 g |

### Example 13

### Soy Pods Reduce Absorption of Dietary Fat in Mice

### Methods

Eight hundred (800) soy plants (variety Pioneer 95Y61) were planted and grown on the LSU AgCenter Dean Lee Research and Extension Center, Alexandria, La. One hundred twenty thousand pods were harvested at the reproductive stage 6 (R6) or when the green pod contains seeds (beans) that have filled the pods. Pods were collected into plastic bags that were sealed, placed on ice, transported to a laboratory, and placed at 4° C. overnight. Each bag of soy pods was washed 3-times with sterile distilled water the next morning.

Pods containing seeds were thinly sliced by placing pod in the food pusher of a food processor (KitchenAid® Model KFP720WH1) that is modified with a 20 ml syringe in the center to hold the pod vertical about 2 mm from the disc-slicing blade. The thin cross sections of pod tissue were transferred as a single layer onto cafeteria trays (9 in×12 in) containing a paper towel that is presoaked with 80 ml distilled sterile water.

Tissues from about 50,000 pods were immediately transferred to gallon size plastic bags that were sealed and placed in a freezer (−80 C). This preparation provided unactivated soy pod sections. The remaining pod cross-sections, were placed on similar trays, and then positioned into Rubbermaid Roughneck containers (10 in height×11 in wide×11 in deep) using Petri dishes as spacers between each tray. Two 150×15 mm Petri dishes were filled with saturated potassium chloride and placed below the bottom cafeteria tray to fix the humidity at 83% after the container was sealed and placed in the dark for 72 h. After incubation at 22.5° C., tissue was transferred into gallon-sized plastic bags, sealed, and stored at −80° C. These samples provided activated soy pod sections. Unactivated and activated soy pod sections were dried in a lyophilizer for 4 days and then were milled using a Retsch Cutting Mill SM 100 with a 0.5 mm screen to produce unactivated soy pod fiber (USPF) and activated soy pod fiber (ASPF).

High performance liquid chromatography (HPLC) was performed to measure soy isoflavones in ASPF and USPF. 0.2 g powder from each pool was weighed into a 1 ml microfuge tube. Methanol (1 ml) was added and the tubes were sonicated in a water bath for 1 h and centrifuged at high speed for 5 min. Supernatants were filtered and transferred to an autosampler vial for HPLC analysis (Waters 2695 combined with a Waters UV-visible 2996 photodiode array detector; Waters Associated. Milford, Mass.).

Soy compounds were separated using a Luna II C18 reverse phase column (4.6×250 mm; 5 μm; Phenomenex, Torrance, Calif.). Solvent A was 0.1% acetic acid in water and solvent B was 100% acetonitrile. A 20 μl volume of sample was injected and the HPLC was programmed with a flow rate of 1.0 ml/min using 15% B for 8 min, then 58% B in 50 min, then 90% B in 10 min followed by holding at 90% B for 10 min. The spectra were collected between 220 and 400 nm by a photodiode array detector and quantitated using a calibration curve from a glyceollin standard at 285 nm.

Sugars in ASPF and USPF were quantified by HPLC as described by Smiricky et al (5). All other analytical assays of ASPF and USPF were performed by Medallion Labs (Minneapolis, Minn.; www.medallionlabs.com) using the nutrient labeling package as required by the Nutrition Labeling and Education Act (NLEA) for nutritional labeling of food products.

Male C57Bl/6NTac mice were ordered from Taconic Biosciences (http:/www.taconic.com/). This widely used, inbred strain becomes obese when fed a diet rich in sugar and fat. Mice were shipped to PreClinOmics (PreClinOmics, Indianapolis, Ind.) where they were individually housed in shoebox caging with woodchip bedding and maintained on a 12:12 light:dark cycle (lights on at 21:00 h) under standard laboratory conditions with a controlled room temperature (20-21° C.). House water was available ad libitumwn throughout the study. The Institutional Animal Care and Use Committee of PreClinOmics approved the protocol and all procedures. The trained staff at PreClinOmics performed experiments and were masked to the treatments.

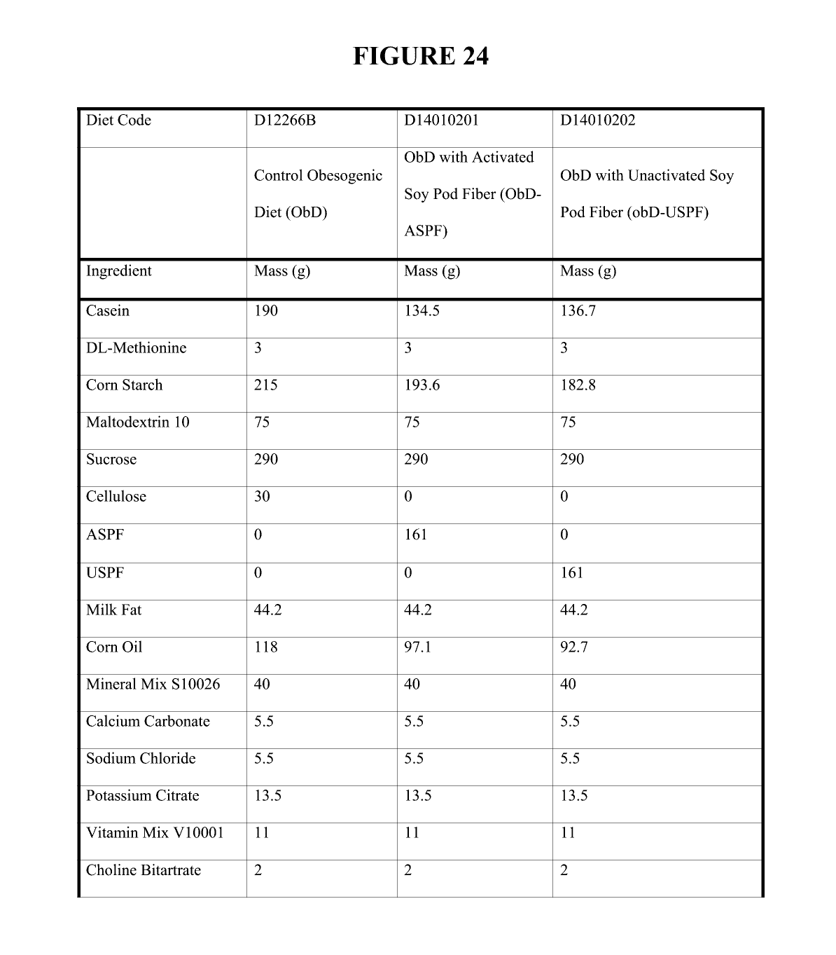

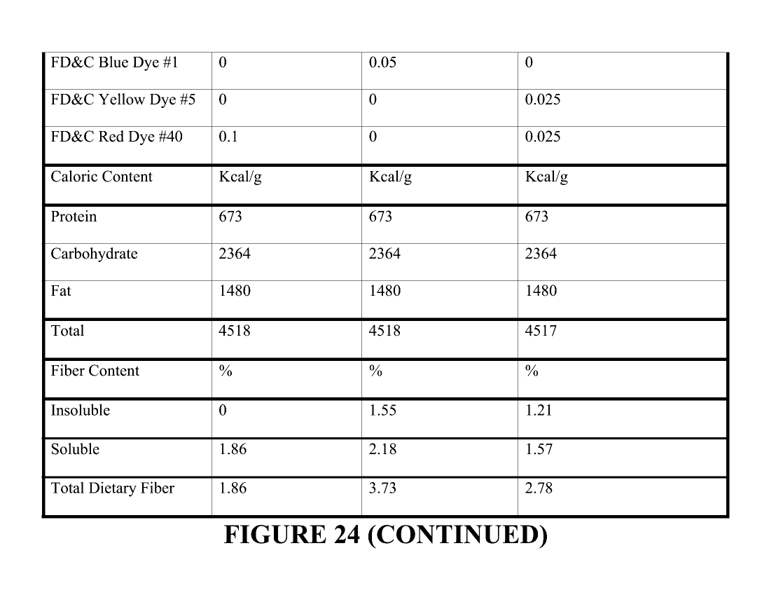

Mice weighing 19.89±0.22 g (mean±SEM) were chosen for the experiment and fed an obesogenic diet (D12266B; Research Diets, New Brunswick, N.J.) on arrival. After 2 weeks of acclimation, 10 mice were randomly assigned to continue consuming D12266B (ObD, obesogenic diet). Ten mice were assigned to consume the ObD, which was modified to contain 15% ASPF (ObD-ASPF, FIG. 24), and 10 mice were assigned to consume the ObD, which was modified to contain 15% USPF (ObD-USPF, Table 1). The 3 diets were isocaloric with the fiber content of both ASPF and USPF matching that of ObD, which contains cellulose instead. Fat and protein content of the soy pod fibers partially replaced corn oil and casein of ObD.

Cages were cleaned on day 9 of the acclimation period to permit daily collection of fecal pellets on days 10-13 (inclusive) of the 2-week acclimation period to serve as baseline samples. Pellets were weighed and stored at −70° C. until assay. At the end of the acclimation period, body composition was measured with high precision and accuracy using NMR-MRI-based technology of the EchoMRI-900 whole body analyzer (Houston, Tex.) without restraint or anesthesia. Mice were then randomly sorted into the 3 diet groups, which were balanced by total body fat mass. Diets were fed for 30 days.

Body weight and food intake were recorded weekly. Fresh chow replaced any remaining at weekly intervals. Clean bedding was added to the cages on d22 to permit feces collection on d24-d27 (inclusive) of treatment. Fecal pellets were weighed and stored at −70° C. until assay. Final body composition was measured on d30 and the mice were then euthanized by decapitation. Trunk blood was collected into a microfuge tube containing EDTA. Following collection, the blood samples were processed for plasma and stored at −20° C. for further analysis. Colons were collected, placed into 10% formalin solution and shipped to Boulder BioPath (Boulder, Colo.) for tissue preparation and histological evaluation.

Plasma interferon-gamma (IFN-γ), interleukin-2 (IL-2), IL-4, IL-5, IL-6, IL-10, keratinocyte chemoattractant growth regulated oncogene (KC/GRO) now called chemokine (C—X—C motif) ligand 1 (CXCL1), and tumor necrosis factor-α (TNF-α) were measured using the V-Plex proinflammatory panel 1 (mouse) assay kit from Meso Scale Discovery (Rockville, Md.).

Colons were cut into two pieces and identified as proximal and distal. Each was embedded in paraffin, sectioned, and stained with hematoxylin and eosin (H&E) according to standard procedures. Procedures and analyses of the sections were performed at Bolder BioPath, Inc (http://www.bolderbiopathDOTcom,), who were blinded to the treatment groups. Because there were no differences between histology of the proximal and distal sections, only data from the proximal sections are presented. A second section was cut and stained with Mayer's mucicarmine to better visualize mucus.

Submucosal edema was quantitated by measuring the distance from the muscularis mucosa to the internal border of the outer muscle layer in a non-tangential area. The extent of macrophage, lymphocyte and polymorphonuclear (PMN) cell infiltrate was assigned severity scores of 0=normal to 5=severe, 76-100% of mucosa affected. Crypt epithelial and remaining gland epithelial loss was scored based on the approximate percent of the mucosa that was affected with 0=none to 5=severe, 76-100% of the mucosa affected. Loss of surface epithelium was scored based on the approximate percent of the mucosa that was affected from 0=none to 5=severe, 76-100% of the mucosa affected. Mucosal thickness was measured in a non-tangential area of the section that best represents the overall mucosal thickness from 0=200 μm (normal) to 5≧650 μm (severe). The number of definite mucosal lymphoid aggregates was recorded. Estimation of goblet cell number was made using the mucicarmine stain and scored from 0=normal to 5=severe, 76-100% loss of goblet cells.

Daily collections of feces for each of the 4 days were pooled and mixed for each mouse providing a baseline- and a final sample for each mouse. Four fecal pellets (˜10 mg/pellet) were homogenized in 100 μl stool diluent (100 ml/L Triton X-100, 6 ml/L Brij® 30 and 0.1 mm/L HCl in isotonic saline) and thoroughly mixed by vortexing. After standing for 30 min at room temperature, samples were centrifuged at 1050×g for 15 min. The supernatants were transferred to auto analyzer cups and they were assayed using a Beckman Coulter AU480 chemistry analyzer with kits to measure plasma levels of TG, lactate, glucose, and cholesterol. The assays were validated for feces by spiking standards.

Fecal pH was performed after mixing dried (lyophilized) fecal samples (4 pellets) with water using a wt:vol ratio of 100:1 (mg/ml). An Orion Research EA-940 pH meter (Thermo Scientific, Waltham, Mass.) with microprobe was inserted into the mixture.

Fecal SCFA were quantified by adding dried feces to H₂O using a 1:10 (wt:vol) ratio. After vortexing thoroughly, 2 mL of 25% m-phosphoric acid was added for each 1 mg feces. The solution was vortexed until a suspension was observed and centrifuged for 10 min at 20,000 g at room temperature. The supernatant was collected and frozen at −20° C. in microfuge tubes until assay. Thawed samples were centrifuged at 10,000 g for 10 min. Concentrations of acetate, propionate, and butyrate were determined in the supernatant using a GC-MS system consisting of an Agilent 7890A (Agilent Technologies, Palo Alto, Calif.) gas chromatograph coupled to an Agilent 5975C mass selective detector (Agilent Technologies, Palo Alto, Calif.) and a fused-silica capillary column with a free fatty acid phase (DB-FFAP 125-3237, J&W Scientific, Agilent Technologies, Palo Alto, Calif.). Helium was supplied as the carrier gas at a flow rate of 14.4 ml/min. The initial oven temperature was 90° C., maintained for 0.5 min, raised to 150° C. at 15° C./min, raised to 170° C. at 5° C./min, and finally increased to 205° C. at 20° C./min. The injector temperature was 250° C. The injection was made in split mode (10:1) with an injection volume of 1 ml. The detector was operated in electron impact ionization mode (electron energy 70 eV), scanning 40-250 m/z range. The temperature of the ion source, quadrupole, and interface were 230, 150, and 280° C.

The internal standard, 2-ethylbutyric acid, was spiked into the supernatant at a final concentration of 1 mM and the supernatant was injected in the GC-MS for analysis. Calibration curves were obtained for each SCFA using the standard SCFA mixture (cat #46975-U, Supelco, Bellefonte, Pa.). Use of extracted ion chromatograms for area calculation and quantification reduced the possibility of misinterpreting overlapping peaks. A characteristic target ion, with the highest m/z value that is relatively abundant was selected for each compound: acetate m/z 60, propionate m/z 74, and butyrate m/z 73. Quantification was accomplished by measuring the peak areas for acetate, propionate, and butyrate relative to 2-ethylbutyric acid (target ion at m/z 88).

1.0 ml of 75% ethanol was added to 50 mg of dried feces, which was first pulverized with a glass rod. The mixture was incubated at 50° C. for 2 h. Incubates were then centrifuged at 1050×g for 10 min and 100 μl of supernatant was added to 500 μl phosphate buffered saline solution (PBS) and vortexed. Samples were assayed using the Crystal Chem mouse total bile acids kit (Downers Grove, Ill.) according to the manufacturer's instructions. A 1:5 75% ethanol:PBS was used as a blank.

About 30 mg of dried mouse fecal pellets were weighed and 5 ml of 0.1% Tween 20 in PBS was added for each 100 mg feces. The pellets rehydrated for 30 min and the microfuge tubes were shaken vigorously by hand and then vortexed to suspend the material. After settling for 10 min. the suspension was centrifuged for 10 min in a microfuge at 1050×g. At least 300 μl supernatant was transfer to a 1 ml microfuge tube for the assay. sIgA was measured with the enzyme-linked immunosorbent assay kit from Cloud-Clone Corp. (SEA641Mu, Houston, Tex.) according to the manufacturer's instructions.

Gross energy content of the feces was measured using an isoperibol calorimeter (Parr Instrument model 6200, Parr Instruments, Moline, Ill.) and mineral oil as a standard. A mineral oil spike (0.2 ml, ˜0.16 g) was pipetted and weighed into a sample cup covering as much of the sample surface as possible. About 5 dried mouse fecal pellets (˜0.040 g) were added and weights of sample and oil were recorded. An ignition wire was prepared and inserted into oil containing the sample and placed into the bomb canister. After filling the sealed canister with oxygen, it was lowered into the bucket containing 500 ml water. The ignition wires were connected to the terminals on the canister bomb head and the instrument was programmed to ignite. Caloric content of the sample was calculated in cal/g feces after correcting for the calorific value of the oil spike.

16s libraries were constructed and taxonomy analyses were performed by Cofactor Genomics (http://cofactorgenomics.com/) who were blinded to the treatment groups. Briefly, genomic DNA (10 ng) was amplified with primers flaking V4 variable region of 16s ribosomal gene. Following amplification, DNA was AMPure XP bead purified. Library quality was assessed and a multiplexed sample library was made by qPCR quantification of individual samples that were pooled in an equimolar ratio. Samples were diluted to a 10 nM stock solution and prepared per Illumina recommendations for sequencing.

Cluster generation and sequencing were performed according to the cluster generation manual and sequencing manual from Illumina (Cluster Station User Guide and Genome Analyzer Operations Guide). Base calls were generated using Casava 1.8.2 (Illumina), and the resulting demultiplexed sequence reads were filtered for low quality.

The Mothur (www[DOT]mothur.org) software was used to build contigs from the raw sequencing reads, and to filter for size and chimeras following the MiSeq SOP (http://www.mothur[DOT]org/wikiMiSeq_SOP) through chimera removal.

Taxonomic assignment was done on the resulting contains with the RDP classifier (https://github.com/rdpstaff/[SLASH]classifier) in multi-sample mode at a level of 0.5.

Results are expressed as mean±1 standard error (SEM). Data were analyzed using a linear model and one-way analysis of variance (ANOVA) on untransformed data (JMP 11.2.0, SAS Institute Inc: Carry, N.C.). Multiple paired Student's t-test comparisons were used when data for baseline values (PreRx) to final values (PostRx). Comparisons to a control value (ObD) were made using Dunnett's method when only terminal data for a variable were available. Comparisons for each pair used Student's T-test that was adjusted for multiple comparisons. Significance for all tests was set at p<0.05.

### Data

ASPF and USPF contained about 50% carbohydrate but total dietary fiber content contributes a majority of that (Table 4). About 30% is protein and 15% fat. The activation process was designed to stimulate glyceollin biosynthesis (Table 4, FIG. 1) but we also observed that this process eliminated free sucrose (ASPF 0 mg/g, USPF 20.2 mg/g) and greatly decreased both free glucose content (ASPF 6.2 mg/g, USPF 14.1 mg/g) and free fructose content (ASPF 2.5 mg/g, USPF 7.3 mg/g) (Table 5).

TABLE 4
Macronutrient and Glyceollin content of ASPF
and USPF. Total glyceollin is the sum of glyceollin
I, glyuceollin II and glyceollin III.

| Analyte | ASPF | USPF |
|---|---|---|
| Carbohydrates (%) | 47.9 | 47.5 |
| Absorbable carbohydrates (%) | 13.3 | 20.0 |
| Insoluble fiber (%) | 34.6 | 27.5 |
| Soluble fiber (%) | 4.1 | 3.8 |
| Protein (%) | 30.0 | 28.8 |
| Fat (%) | 13.4 | 16.1 |
| Calories (Cal/g) | 432 | 450 |
| Calories (Insol fiber subtracted) Cal/g | 294 | 340 |
| Total glyceollin (μg/g)¹ | 175 | 0 |

TABLE 5
Saccharide content of ASPF and USPF.

| Sugar Content (μg/g) | ASPF | USPF |
|---|---|---|
| Fucose | 21.0 | 15.2 |
| Arabmose | 608.0 | 57.7 |
| Rhamnose | 51.2 | 0 |
| Galactose | 754.1 | 506.9 |
| Glucose | 6231.5 | 14057.9 |
| Sucrose | 0 | 20227.1 |
| Xylose (μg/g) | 250.7 | 0 |
| Mannose (μg/g) | 0 | 0 |
| Fructose (μg/g) | 2542.6 | 7266.7 |
| Total free sugar (mg/g) | 10.5 | 42.1 |

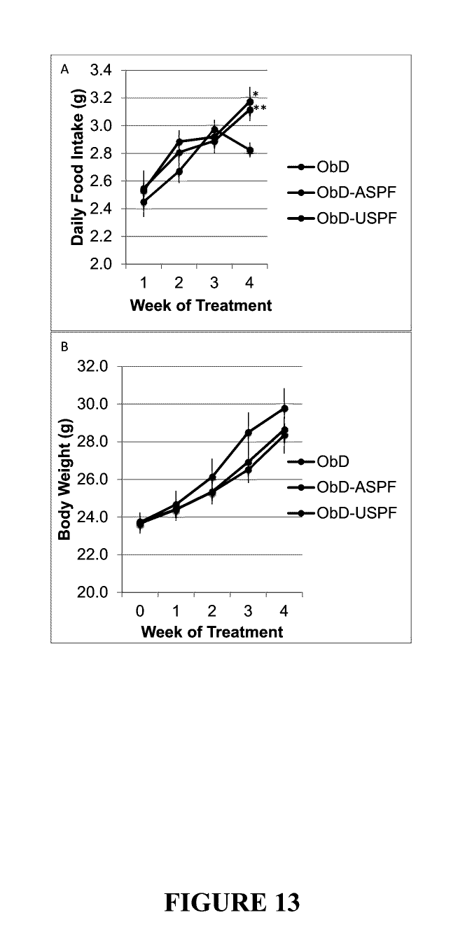

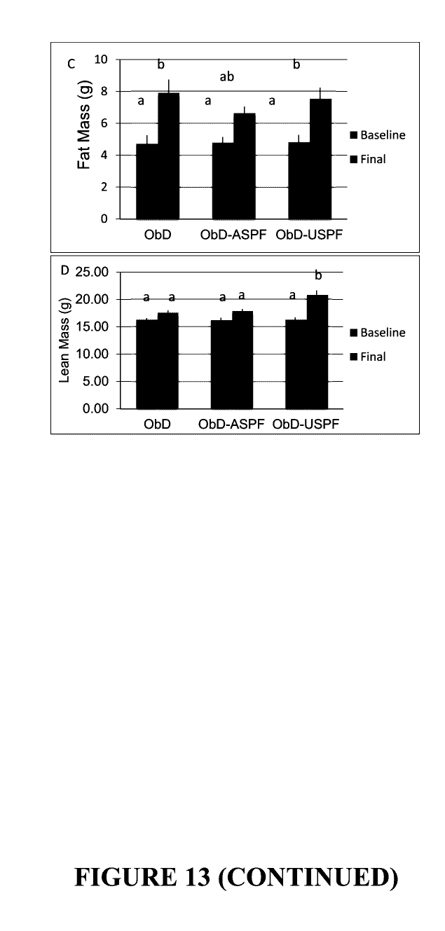

Supplementing ObD with either ASPF or USPF was not appetite aversive. Daily intake increased during the first 3 weeks when consuming all diets (FIG. 13A). Food intake appeared to remain at that level for the final week of mice assigned to the ObD diet, but continued at a constant rate for mice consuming either ObD-ASPF or ObD-USPF. Cumulative food intake over the 4 weeks was not statistically different when comparing groups. Despite constant consumption of the isocaloric diets, mice assigned to either the ObD-ASPF or ObD-USPF diets tended to gain weight at a slower rate than mice fed the ObD (FIG. 13B). Mice consuming ObD gained 5.9±0.7 g, which was comprised of a statistically significant increase in fat mass (3.2±0.8 g, FIG. 13C). Mice ingesting ObD-ASPF gained the least body mass (4.4±0.5 g) that included only 1.8±0.7 g fat and 2.7±0.5 g lean mass (FIG. 13D). Mice eating ObD-USPF gained 5.3±0.4 g, which was primarily lean mass (4.5±1.0 g lean and 2.7±0.5 g fat mass).

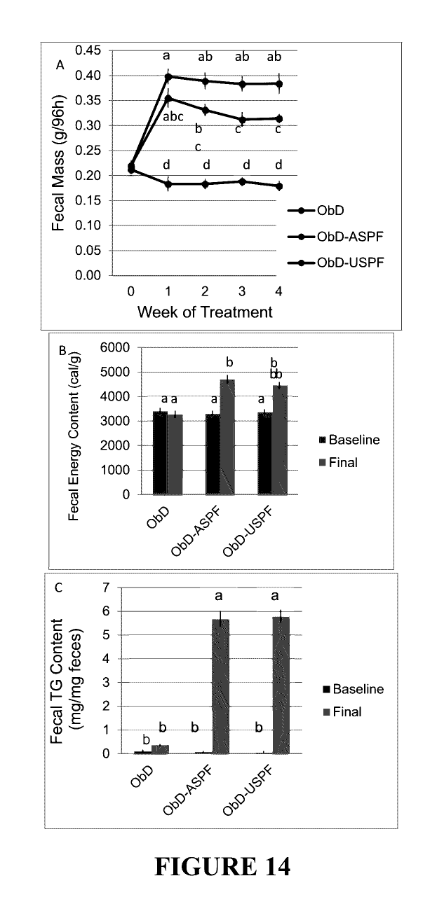

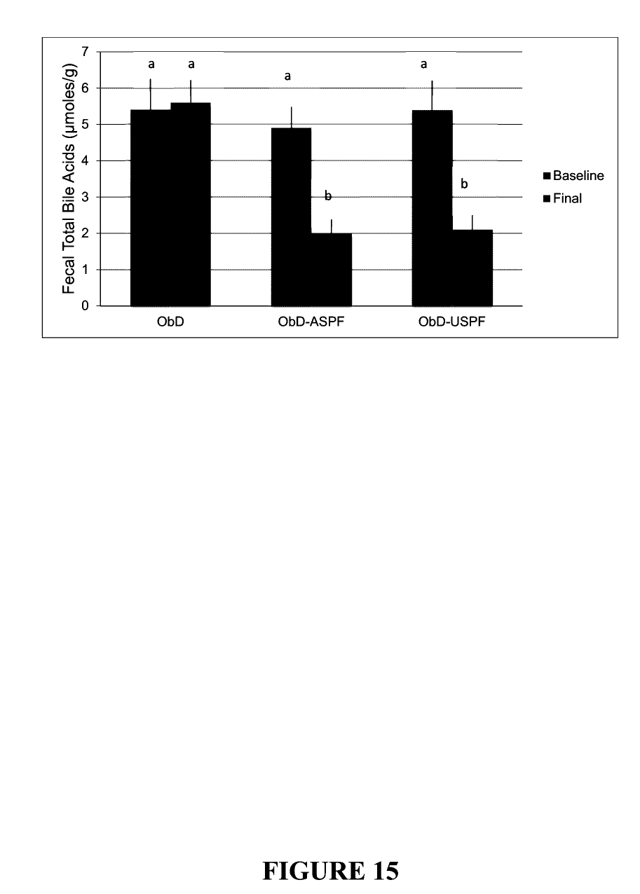

Some of the reduced energy gain is accounted for as loss in the feces. Fecal output was doubled for mice offered the fiber-supplemented diets (FIG. 14A). Fecal mass collected from mice consuming ObD-ASPF was significantly greater than that from mice eating ObD-USPF during the final 2 weeks. Additionally, the energy content of fecal pellets was greater (p<0.05) in feces from both test diets during the final week (FIG. 14B). Some of the energy in feces was from triglycerides (FIG. 14C) that was increased almost 50-fold by the supplemented diets. Fecal pellets were solid for all groups with no detectable oil leakage. Reduced absorption of TG may be a consequence of reduced bile acid levels in the lumen as detected in the feces (FIG. 15).

Bile acids are transformed to bile salts by some intestinal bacteria and are toxic to others. Bile acid production and secretion by the liver are stimulated by dietary fat so we characterized the microbiota profile in feces of mice consuming the 3 diets. We observed phylum-level shifts in the microbiota composition of feces from mice fed the 3 diets. The shifts between the 2 dominant phyla Bacteroidetes (70% of community) and Firmicutes (25% of community) tended to increase and decrease in abundance, respectively, when ObD was supplemented with ASPF and USPF but those differences were not statistically different. However, there was a significant (p<0.05) decrease in abundance of Verrucomicrobia species in feces of mice fed both ASPF and USPF as well as a decrease (p<0.05) in abundance of Deferribacteres species in feces of mice fed the USPF supplemented diet.

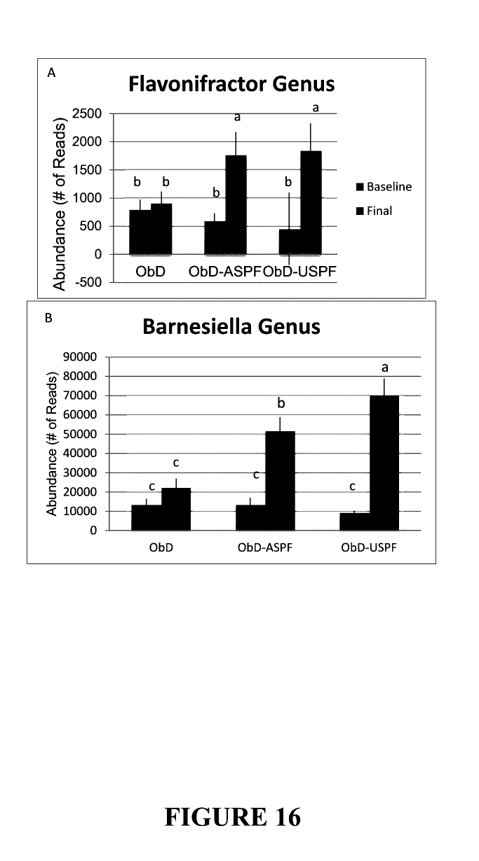

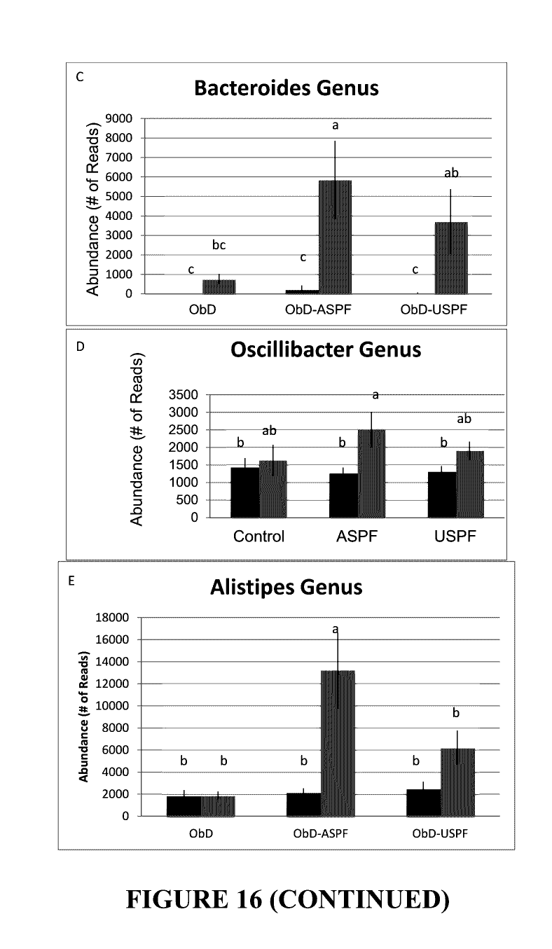

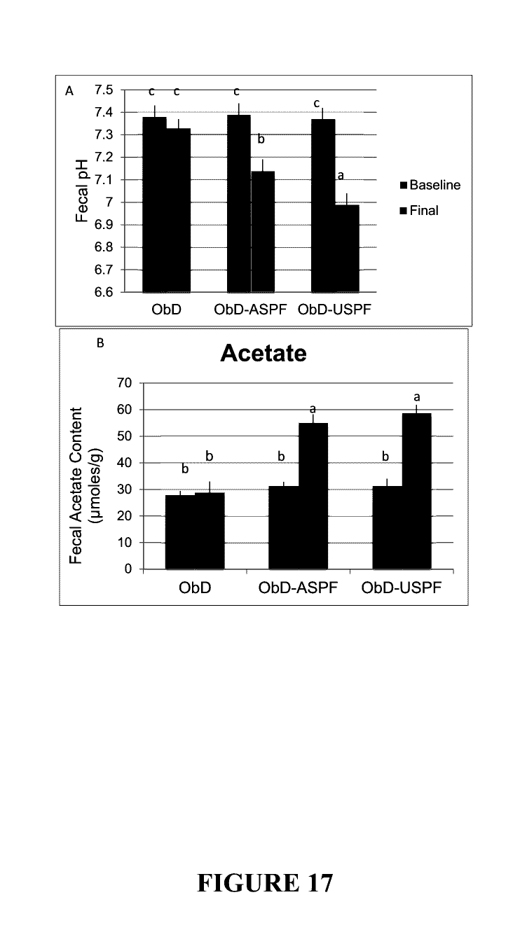

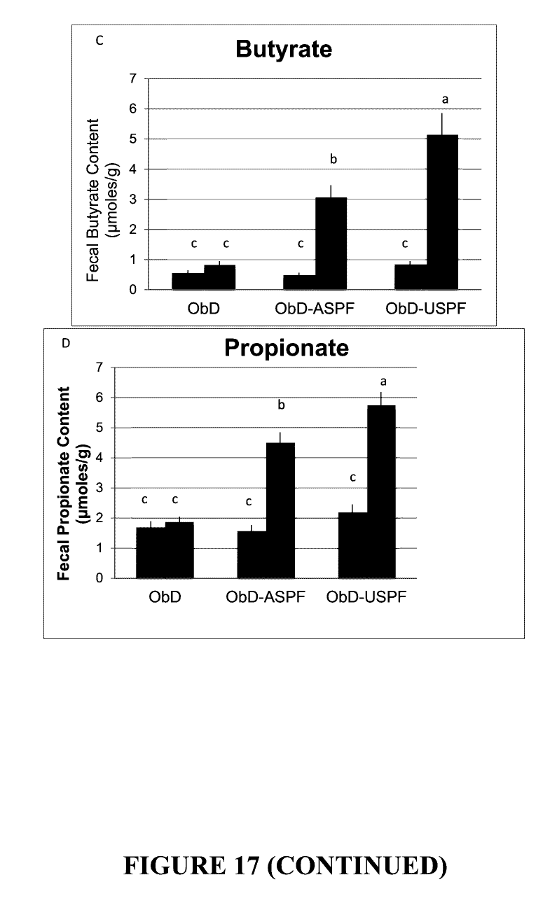

Because bile acids in feces were reduced and they are toxic to some species, we explored which genera may have an increased abundance of species. Species in 3 genera; Flavonifractor (FIG. 16A), Barnesiella (FIG. 16B), and Bacteroides (FIG. 16C) were significantly increased by consumption of both ObD-ASPF and ObD-USPF. Oscillibactor—(FIG. 16D) and Alistipes- (FIG. 16E) species were only stimulated by ObD-ASPF. In addition to decreased levels of bile acids in the microbiome, some species in these genera are capable of fermenting carbohydrates and are increased in abundance by administering prebiotics to the diet. Therefore, we explored for evidence of fermentation using fecal biomarkers. We observed a decrease in fecal pH (FIG. 17A) with an increase in fecal content of acetate (FIG. 17B), butyrate (FIG. 17C), and propionate (FIG. 17D). The apparent increase in fermentation was greater for ObD-USPF than ObD-ASPF.

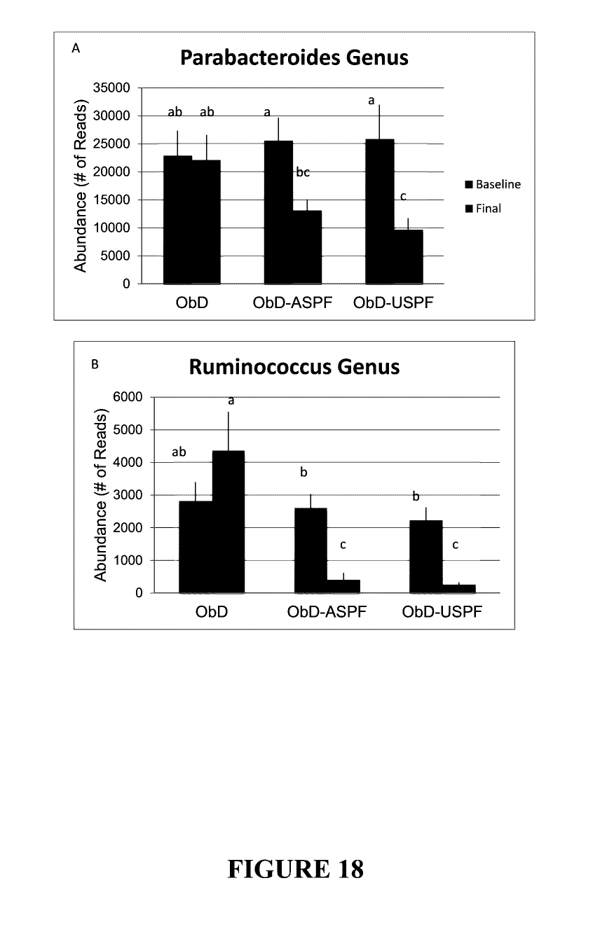

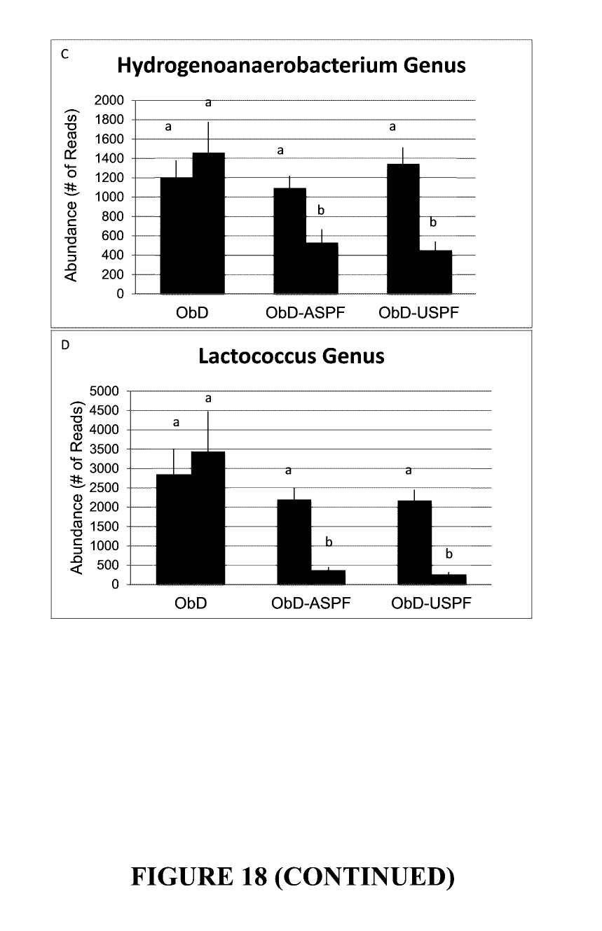

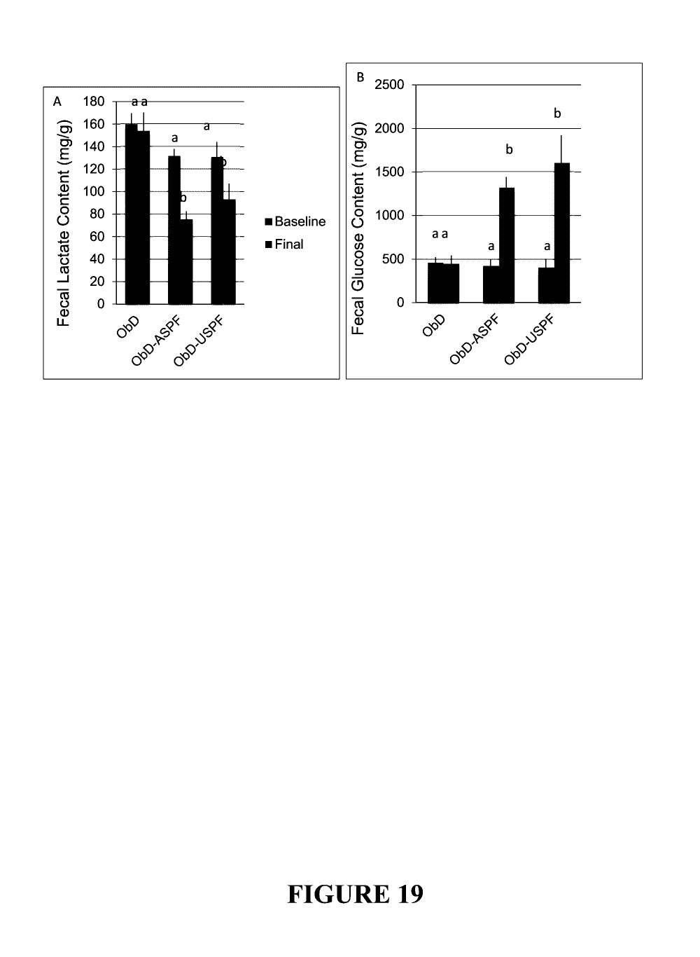

Species that were significantly decreased in abundance belong to 6 genera. Species in Parabacteroides (FIG. 18A), Ruminococcus (FIG. 18B). Hydrogenoanaerobacterium (FIG. 8C) and Lactococcus (FIG. 18D) genera were significantly decreased in both ObD-ASPF and ObD-USPF groups. Lactococcus species utilize glucose in the biome to create lactate. Consistent with a decrease in abundance of Lactoccus species is the observation that fecal lactate content was significantly decreased (FIG. 19A) and fecal glucose content increased (FIG. 19B).

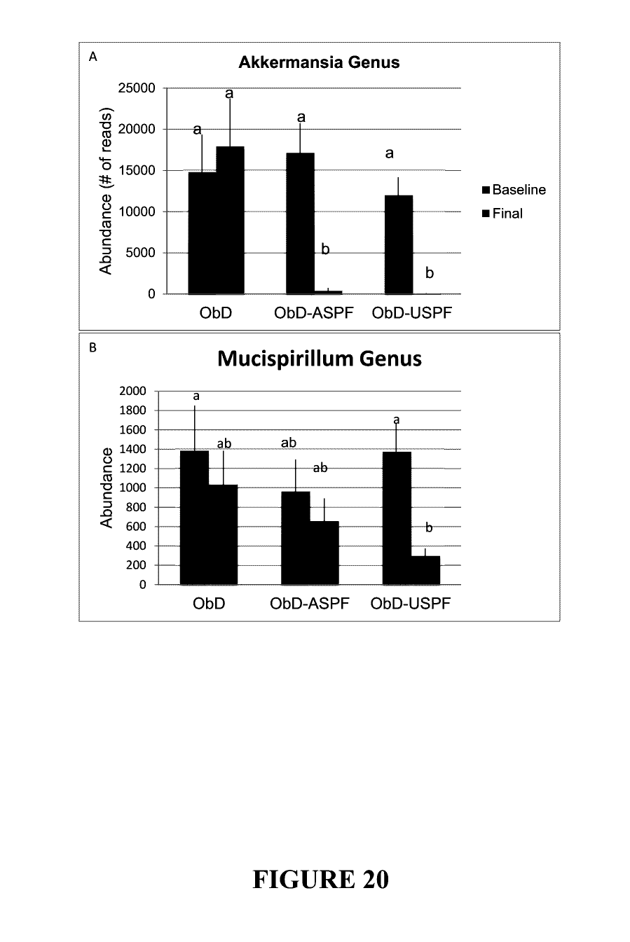

Species of 2 other genera that were significantly decreased in abundance are considered mucolytic species. Akkermanisia species were significantly reduced in abundance by both ObD-ASPF and ObD-USPF (FIG. 20A). Mucispirillum species were significantly reduced in abundance in feces of mice only consuming ObD-USPF (FIG. 20B).

Colonization of mucus by different mucolytic species may be crucial with respect to colon mucosa. Therefore, we studied colon histopathology. Significantly increased scores for inflammation and neutrophil were only observed in mice consuming the ObD-USPF (Table 6, FIG. 1) suggesting the decrease in abundance Mucispirillum species withdraws some mucosal protection.

TABLE 6
Histopathology of the proximal colon in mice after consuming ObD,
ObD-ASPE or ObD-USPF for 30 days. 10 sections from each group were selected.

| | | Histopathology Scores | | | | | | Muci- |
|---|---|---|---|---|---|---|---|---|
| | Edema | Inflam- | Gland | | PMN | Neutrophil | Lymphoid | carmine |
| | (μm) | mation | Loss | Erosion | (%) | Score | Score | Score |
| ObD | 0.0 ± 0.0 | 0.4 ± 0.08 | 0.0 ± 0.0 | 0.0 ± 0.0 | 7 ± 2 | 0.0 ± 0.01 | 0.4 ± 0.22 | 0.0 ± 0.0 |
| ObD-ASPF | 0.0 ± 0.0 | 0.5 ± 0.12 | 0.0 ± 0.0 | 0.0 ± 0.0 | 7 ± 2 | 0.0 ± 0.01 | 0.2 ± 0.13 | 0.0 ± 0.0 |
| ObD-USPE | 0.0 ± 0.0 | 0.7 ± 0.08* | 0.0 ± 0.0 | 0.0 ± 0.0 | 10 ± 0 | 0.1 ± 0.01* | 0.8 ± 0.25 | 0.0 ± 0.0 |

PMN is % of polymorphonuclear cells and mucicarmine is the abundance of mucin.
*p < 0.05.

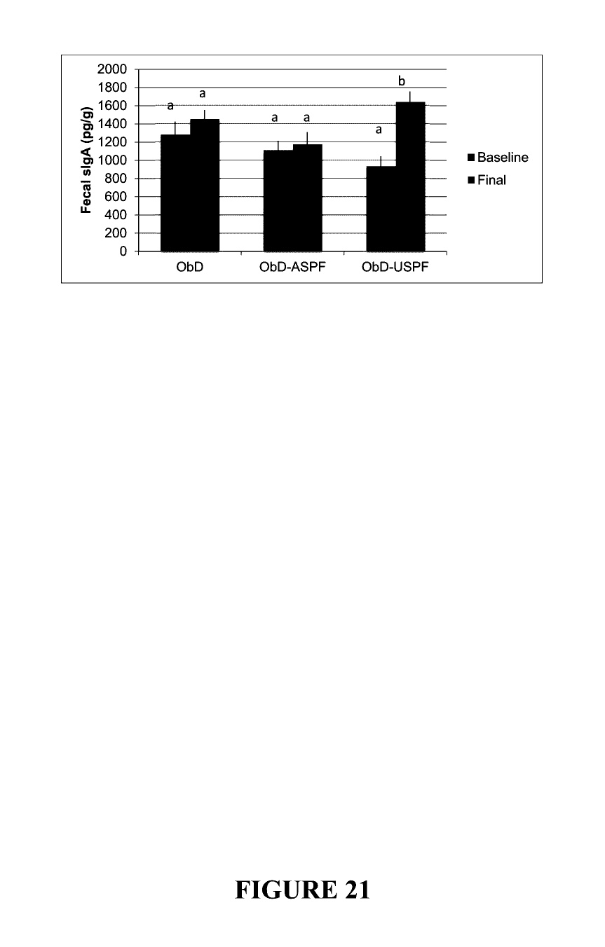

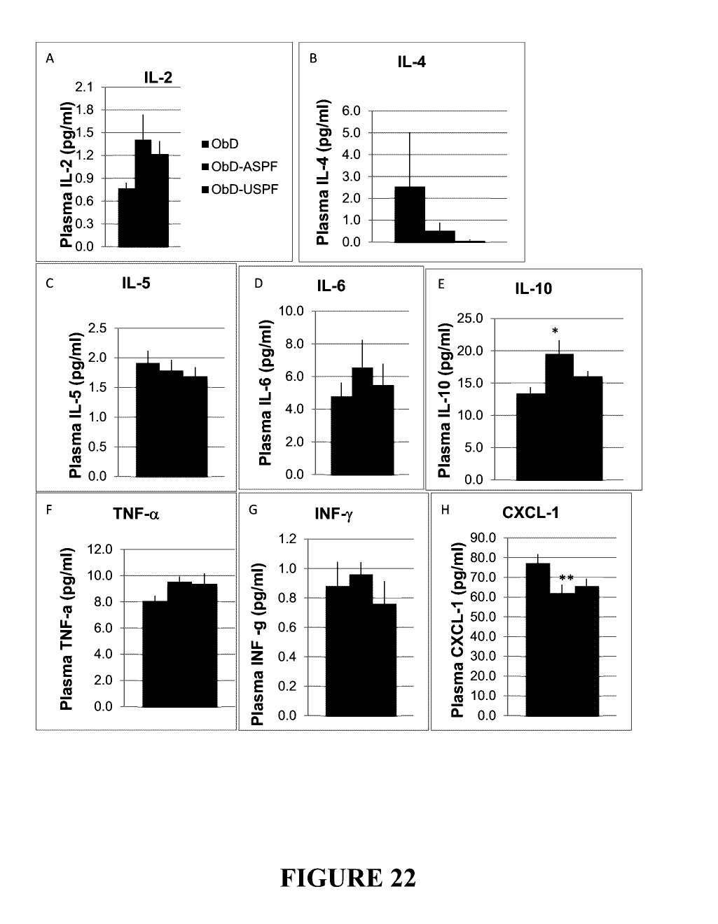

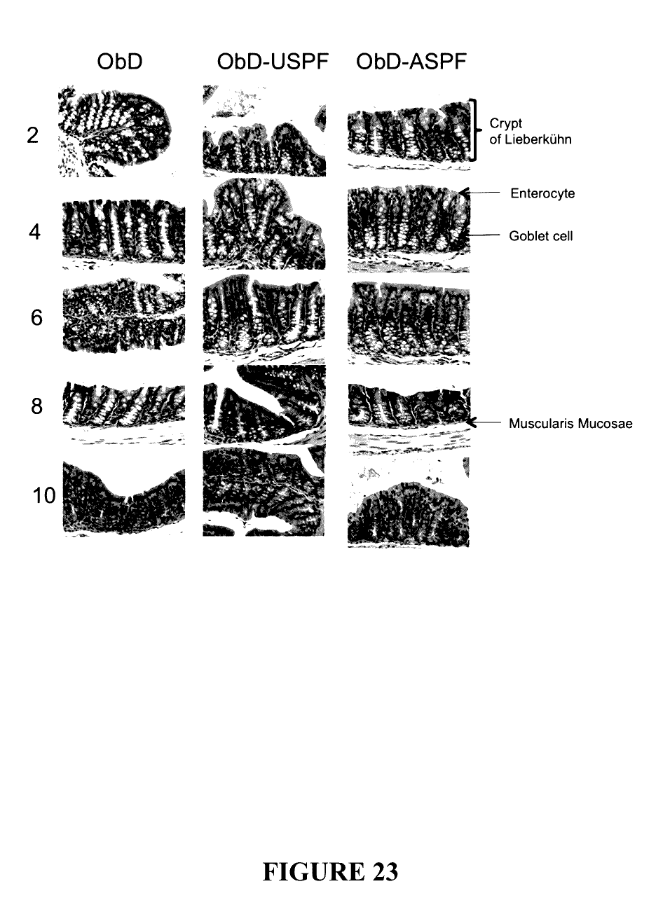

Consistent with colon inflammation that was mediated by the ObD-USPF diet, there was an increase in fecal sIgA content (FIG. 21). This was not observed in feces of ObD-ASPF fed mice suggesting that the glyceollins may protect against inflammation. Protection of colon inflammation provided by ASPF was consistent with changes observed in the plasma proinflammatory panel data (FIG. 22). An increase in plasma anti-inflammatory IL-10 (FIG. 22E) and a decrease in circulating CXCL1 (FIG. 22H) that acts as a chemokine to recruit leukocytes was observed in mice consuming ObD-ASPF.

### Overview

Until recently, the community of GI microbes remained largely unstudied (6,7). Insights into how diet, the GI microbiome, and human host interact during different physiological and pathological states are unveiling how these relationships support health or trigger disease. The GI microbiome is comprised of GI microbia, its metabolic secretome, and a solution of chime, which are bounded by intestinal mucosa. Diet is the principal element that determines the character of the GI microbiome. Thus a caloric dense diet—GI microbiome partnership may predispose an individual to metabolic disorders such as obesity and diabetes by modulating nutrient acquisition, energy harvest, GI immunity, metabolic pathways, and GI hormones. Dietary habits are difficult to change so we developed a GIMM from soybean pods containing glyceollins as a dietary intervention. We demonstrate that supplementing a diet rich in fat and sugar with the GIMM, decreased dietary fat uptake by reducing the bile acid pool. This improved body composition with protection from inflammation.

Fruit of the soy plant is a pod containing seeds or soybeans, which have very low levels of dietary fiber. We speculated that the pod could greatly contribute novel dietary fiber and included that in the GIMM. We harvested soy pods containing soybeans during a growth phase when they are typically harvested for edamame. Part of the material was activated (ASPF) to produce the glyceollins, which are soy molecules with antioxidant and antibiotic activity (2) that are poorly bioavailable (3,4). The remainder was unprocessed (USPF). This is the first demonstration that glyceollin can be produced in large scale by only slicing the entire pod. Previous methods utilize an incubation of the soybean with a fungus (2). Surprisingly, the activation process eliminated sucrose and greatly reduces both glucose and fructose content. We believe that environmental microbiota was present during activation, which fermented the free sugars and could have also stimulated glyceollin synthesis.

The macronutrient content of both ASPF and USPF were analyzed before developing the rodent chow so that ObD, ObD-ASPF and ObD-USPF would be balanced and isocaloric. Cellulose was added to ObD to account for insoluble fiber content of ASPF and USPF. Corn oil and casein of ObD was partially replaced by soy oil and soy protein of the test diets. Addition of either ASPF or USPF to the ObD did not alter food intake suggesting that there is no food adversive effects. In fact, there was a trend for food intake to be increased by the unique diets. Rather than an accompanying trend for increase in weight gain, we observed a trend for reduced body weights in the ObD-ASPF- and ObD-USPF-fed groups. The former gained the least body mass that was not statistically different than at baseline. However, body composition was significantly altered. Mice consuming ObD gained significant body fat over the 30 d but feeding ObD-ASPF blocked that. Mice fed ObD-USPF accumulated significantly greater fat mass but also a statistically significant increase in lean mass.

At least part of the lack of fat gain can be accounted for by the loss of energy in feces. Fecal output from ObD-ASPF fed mice was about twice that for mice fed ObD and the caloric content of feces was about 1.4-times that from the ObD fed group. Loss of energy in feces was also significantly greater from mice fed ObD-USPF when compared to ObD mice but fecal output was less for this group than it was for ObD-ASPF group during the final week of treatment. Fecal triglyceride content was greater from mice consuming ObD-ASPF and ObD-USPF and accounts for some of the fecal calories that were not absorbed. The loss of TG in the feces was not associated with oil leakage or oily stools as is observed in mice treated with a pancreatic lipase inhibitor. A loss of TG in feces can be a consequence of including flavonoids in a diet (8) but may also be a result of a shift in the GI microbiome. Germ-free mice are inefficient at harvesting energy from dietary fat and excrete 40% more TG in feces than conventionally raised mice (9).

Dietary fat stimulates bile acid synthesis and secretion into the duodenum, a process that is negatively regulated by famesoid X receptors (FXRs) in the liver and GI (10,11). Most primary bile acids are reabsorbed in the ilium to recirculate back in the liver. Those that escape the enterohepatic circulation can be converted to secondary bile acids by GI microbiota in the colon. Both primary and secondary bile acids function as FXR agonists. Recently, production of both primary and secondary muricholic bile acids of mice were shown to function as FXR antagonists, which are increased by presence of the GI microbiota (12). Thus, GI microbiota could alter the balance of bile acid agonists and antagonists at the FXRs. In this study, we observed total bile acid content in feces of ObD-ASPF and ObD-USPF to be significantly reduced as is also observed in germ free mice (12) and mice treated with antibiotics (12). It is possible that a component of the soy protein added to ObD inhibits the bile salt export pump (13). Soy isoflavones are poorly absorbed, secreted into bile (14) and have agonist activity at the FXR (46). Thus, 1 or more soy isoflavone may function as an FXR agonist with poor bioavailability.

Conjugated bile acids are toxic to bacteria and to the intestinal mucosa. However, some microbiota have adapted ability to produce bile salt hydrolase, which benefits microbiota by establishing some resistance to the primary bile acids and also benefits the host from bile acid toxicity (16). Since we observed a decrease in fecal total bile acids we determined if there would be a shift in the fecal microbiota profile. We observed 95% of taxa to reside in the 2 dominate phyla Bacteroidetes and Firmicutes. The relative abundances that we observed were similar to that reported for the same mouse strain consuming the identical ObD diet (17). We observed statistically significant shifts in abundances of 11 genera (5 were increased and 6 were decreased).

The 5 genera that were increased in abundance may be sensitive to bile acids and thus reducing this negative influence in the microbiome could offer a selective advantage to those species. Abundances of Flavonifractor, Barnesiella, and Bacteroides species were increased in feces of both ObD-ASPF and ObD-USPF groups. Abundances of species of Alistipes and Oscillibacter were only increased in feces of mice treated with ASPF. Flavonifractor species are stimulated by the antioxidant epigallocatechin (18), which is a molecule similar to the flavanols in soy. Barnesiella species do not appear to express bile salt hydrolase and blooms of these species can be stimulated by addition of prebiotics (19). Abundances of species in this genus are decreased in irritable bowel syndrome (IBS) (20,21). Abundances of Bacteroides species were almost absent in ObD fed mice but greatly enhanced by addition of ASPF and USPF to the diet. Bacteroides species are decreased with exposure to bile acids (22), decreased in overweight and obese individuals (23) and decreased in patients with liver cirrhosis (24). Soy isoflavones may stimulate a bloom in Bacteroides species that can metabolize isoflavones (25). Alistipes species were only significantly stimulated by ObD-ASPF, suggesting positive influence of the glyceollins. Abundances of phylotypes in this genus are decreased in mice fed lard but stimulated by treatment with metformin (26) or following gastric bypass (27) surgery. Oscillibacter species were also only stimulated by the ObD-ASPF. Species in this genus are decreased in abundance in patients with liver cirrhosis (24) and mice that become diabetic when fed an ObD (28). Collectively, species in these genera appear to be sensitive to dietary fat and likely bile acids. Supplementing the fat diet with ASPF and USPF reverses that. Alistipes and Oscillibacter species appear to be stimulated by glyceollins.

Species that were decreased by ASPF and USPF were in genera Parabacteroides, Ruminococcus, Hydrogenanaerobacterium, Lactococcus, Mucispirillum, and Akkermansia. Parabacteroides are enriched in fecal samples from obese (29), T2D (30,31), autism (32), and consumption of saccharine (33) or resistant starch (34). Administration of metformin decreases abundances of Parabacteroides species (26). Abundances in genera Ruminococcus are increased under conditions of chronic bowel inflammation (35), autism spectrum disorder (36) and IBS (37). Abundances of Hydrogenanaerobacterium are stimulated by ObD (17, 38). Feeding dietary fat increases Lactococcus (17, 26) and that is reversed by metformin (26), prebiotics (19) or vertical sleeve gastrectomy (39). Children with IBS have increased abundances of Lactococcus species (20). Lactococcus species utilize glucose in the biome to create lactate. Consistent with our observation of Lactococcus species abundance decrease by ObD-ASPF and ObD-USPF, was our observations that fecal lactate was decreased and fecal glucose was increased.

Species of Akkerumnsia genus were decreased after feeding either ObD-ASPF or ObD-USPF but species of Mucispirillum were only reduced by ObD-USPF. Species in these 2 genera are mucolytic and thus mucosal colonization may be crucial with respect to human health. Therefore we studied histology of the colon. Only mice consuming ObD-USPF were observed with increased scores for inflammation and neutrophil numbers suggesting the decrease in abundance Mucispirillum species withdraws some protection. Abundance of Mucispirillum species are decreased in feces of mice fed a high fat diet (40) and when colitis is introduced by infection (41). This relationship is less clear for presence of Akkermanisia species, which were dramatically decreased after feeding either ObD-ASPF or ObD-USPF. Colon histopathology of mice consuming the ObD-ASPF appeared normal. We observed no change in mucosal thickness despite the dramatic decrease in species richness. Everard and colleagues demonstrated an increase in mucosal thickness after administering Akkermansia species by oral gavage, suggesting a positive correlation between Akkermansia abundance and mucosal integrity (42). Because Akkermansia species are underrepresented in the GI microbiome, the decrease in abundance that we observed may represent a smaller change than the increased colonization produced by oral gavage. Increases in abundance of Akkermansia are reported in IBS (43), Chinese-(31) but not European-T2D (44), and autism (45). In humans, an omnivore but not vegan diet, produces proatherogenic trimethylamine-N-oxide (TMAO) that may be partially related to presence of Akkermansia (46). Richness of Akkermansia species of mice consuming a high fat diet is decreased by including glucose and saccharine (33) but stimulated by supplementation with fermentable oligosaccharides (19).

Fermentation of carbohydrates is a role for many microbiota and a hallmark of prebiotics. We measured short chain fatty acids and pH in the feces for evidence of fermentation. We observed a decrease in pH with increases in content of acetate, butyrate and propionate, indicating that the fiber in ASPF and USPF was fermentable. Obvious candidates for this fermentation are from the 5 genera that were increased in abundance. These were Barnesiella, Bacteroides, Alistipes, Flavonifractor, and Oscillibacter. In particular, Bacteroides thetaiotaomicron is a species capable of fermenting complex carbohydrates found in yeast walls (47) and could possibly ferment those in the soy pod fibers. Alistipes and Oscillibacter species appear to ferment resistant starch (47) and Barnesiella are stimulated by oligofructans (19). There is no evidence for fermentation by Flavonifractor species.

Mucosal tissues in the GI tract harbor more cells of the immune system than all the secondary lymphatic tissues combined. One prominent feature of mucosal immunoresponses is the production of secretory IgA, which requires microbial colonization in the intestine. Secretory IgA has long been known to act as a mucosal barrier to infection and inflammation by preventing attachment of bacteria viruses, fungi, parasites and food particles to epithelial cells. We observed an increase in fecal sIgA levels in only the mice consuming the ObD-USPF. This was the diet associated with a colon inflammation and a decrease abundance of Mucispirillum species but no increases in abundances of Alistipes or Oscillibacter species as were observed in mice consuming ASPF. Furthermore, plasma levels of the anti-inflammatory IL-10, which inhibits inflammatory responses, were statistically increased and CXCL1, which acts as a chemokine to recruit leukocytes, was decreased by only the ASPF-supplemented ObD. The primary difference between the 2 diets is that ASPF contains glyceollins that are known to be anti-inflammatory (48.49) and to have low bioavailability.

In summary, we developed a novel food ingredient from soy pods that acts as a GIMM to hinder absorption of dietary fat and sugar in mice. Components of the GI microbiome that were modified included shifts in abundances of microbiota in 11 genera, decreases in bile acid excretion, increases in fecal energy, increases in fecal glucose and TG with decreased fecal lactate. When the soy pod tissue is activated to contain glyceollins, the GIMM is anti-inflammatory. We think that the combination of glyceollins with other macro- and micro-nutrients in ASPF could offer an ideal GIMM for individuals who regularly consume diets rich in fat and sugars but lacking sufficient dietary fiber and antioxidants. Although we believe that the combination of glyceollin with other factors in the soy preparation is important for the collective microbiome shift, the data also suggest that glyceollins may be useful in treating and preventing colon inflammation. More studies in animal models of diet-induced dysfunctional GI biome are needed to predict which indication is best for this GIMM. Possible indications are nonalcoholic fatty liver disease that could lead to nonalcoholic steatohepatitis and cirrhosis, obesity, T2D, IBS, IBD, autism, chronic constipation.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### REFERENCES

1. Gu L, Gu W. Characterisation of soy isoflavones and screening for novel malonyl glycosides using high-performance liquid chromatography-electrospray ionisation-mass spectrometry. Phytochemical Analysis 2001; 12:377-382.
2. Zimmerman M C, Tilghman S L, Boud S M, et al. Glyceollin I, a novel phytoalexin isolated from activated soy. J. Pharmacol. Exper. Ther. 2010, 332, 35-45.
3. Boué S M, Isakova I A, Burow M E, et al. Glyceollins, soy isoflavone phyoalexins, improve oral glucose disposal by stimulating glucose uptake. J Agric Food Chem 2012; 60:6376-82.
4. Wood C E, Clarkson T B, Appt S E, et al. Interactive effects of soybean glyceollins and estradiol in the breast. Nutr Can 2006; 56:74-81.
5. Smiricky-Tjardes M R, Grieshop C M, Flickinger E A, et al. Dietary galactooligosaccharides affect ileal and total-tract nutrient digestibility, ileal and fecal bacterial concentrations, and ileal fermentative characteristics of growing pigs. J Anim Sci 2003; 81:2535-45.
6. The human microbiome consortium. Structure, function and diversity of the healthy human microbiome. Nature 2012; 486:207-14.
7. The metaHIT consortium. An integrated catalog of reference genes in the human gut microbiome. Nature Biotech 2014; 32:834-41.
8. Jung U J, Lee M-K, Park Y B, et al. Effect of citrus flavonoids on lipid metabolism and glucose-regulating enzyme mRNA levels in type-2 diabetic mice. Int J Biochem Cell Biol 2006; 38:1134-1145.
9. Rabo S. Membrez M, Bruneau A, et al. Germ-free C57Bl/6J mice are resistant to high-fat-diet-induced insulin resistance and have altered cholesterol metabolism. FASEB J 2010; 24: 4948-4959
10. Goodwin B. Jones S A. Price R R, et al. A regulatory cascade of the nuclear receptors FXR, SHP-1, and LRH-1 represses bile acid biosynthesis. Mol Cell 2000; 6: 517-26.
11. Lu T T, Makishima M, Repa J J. et al. Molecular basis for feedback regulation of bile acid synthesis by nuclear receptors. Mol Cell 2000; 6: 507-15.
12. Savin S I, Waström A, Felin J, et al. Gut microbiota regulates bile acid metabolism by reducing the levels of tauro-beta-muricholic acid, a naturally occurring FXR antagonist. Cell Metab 2013; 17(2):225-35.
13. Hashidume T, Sasaki T, Inoue J, Sato R. Consumption of soy protein isolate reduces SERBP-1c and lipogenic gene expression in wild-type mice, but not in FXR-deficient mice. Biosci Biotechnol Biochem 2011; 75(9): 1702-7.
14. Larkin T, Price W E, Astheimer L. The key importance of soy isoflavone bioavailability to understanding health benefits. Critical Rev Food Sci and Nutr 2008; 48(6): 538-552.
15. Rickettsa M-L, Moorea D D, Banzb W J, et al. Molecular mechanisms of action of the soy isoflavones includes activation of promiscuous nuclear receptors. A review. J Nutr Biochem 2005; 16:321-30.
16. Jones B V, Beglev M, Hill C, et al. Functional and comparative metagenomic analysis of bile salt hydrolase activity in the human gut microbiome. PNAS 2008; 105(36): 13580-85.
17. Parks B W, Nam E, Org E, et al. Genetic control of obesity and gut microbiota composition in response to high-fat, high sucrose diet in mice. Cell Metab 2013; 17:141-152.
18. Takagaki A, Kato Y. Nanjo F. Isolation and characterization of rat intestinal bacteria involved in biotransformation of (−)-epigallocatechin. Arch Microbiol 2014; 196(10)681-95.
19. Everard A, Lazarevic V, Derrien M, et al. Responses of gut microbiota and glucose and lipid metabolism to prebiotics in genetic obese and diet-induced leptin-resistant mice. Diabetes 2011; 60:2775-86.
20. Saulnier D M, Riehle K, Mistretta T A, et al. Gastrointestinal microbiome signatures of pediatric patients with irritable bowel syndrome. Gstroenterol 2011; 141(5): 1782-91.
21. Jeffery I B, O'Toole P W, Öhman L. et al. An irritable bowel syndrome subtype defined by species-specific alterations in faecal microbiota. Gut 2012; 61(7):997-1006.
22. Islam K B, Fukiya S, Hagio M, et al. Bile acid is a host factor that regulates the composition of the cecal microbiota in rats. Gastroenterol 2011; 141(5):1773-81.
23. Femandes J, Su W, Rahat-Rozenbloom S, et al. Adipocity, gut microbiota and fecal short chain fatty acids are linked in adult humans. Nutr Diabetes 2014; 4 e121.
24. Qin N, Yang F, Li A, et al. Alterations of the human gut microbiome in liver cirrhosis. Nature 2014; 513:59-64.
25. Renouf M, Hendrich S. Bacteroides uniformis is a putative bacterial species associated with degradation of the isoflavone genistein in human feces. J Nutr 2011; 141(6): 1120-26.
26. Shin N-R, Lee J-C. Lee H-Y, et al. An increase in the Akkermansia spp. Population induced by metformin treatment improves glucose homeostatis in diet-induced obese mice. Gut 2014; 63:727-35.
27. Liou A P, Paziuk M, Luevano J-M, et al. Conserved shifts in the gut microbiota due to gastric bypass reduce host weight and adiposity. Sci Transl Med 2013; 5:178ra41.
28. Serino M, Luche E, Gres S, et al. Metabolic adaptation to a high-fat diet is associated with a change in the gut microbiota. Gut 2012; 61:543-53.
29. Ridaura V K, Faith J J, Rey F E, et al. Gut microbiota from twins discordant for obesity modulate metabolism in mice. Science 2013; 341(6150):1241214.
30. Wu X, Ma C, Han L, et al. Molecular characterization of the fecal microbiota in patients with type II diabetes. Curr Microbiol 2010; 61:69-78.
31. Qin J, Li Y, Cai Z, et al. A metagenome-wide association study of gut microbiota in type 2 diabetes. Nature 2012; 490:55-60.
32. Finegold S M. Dowd S E, Gontcharova V. et al. Prosequencing study of fecal microlora of autistic and control children. Anaerobe 2010; 16:444-53.
33. Suez J, Korem T, Zeevi D, et al. Artificial sweeteners induce glucose intolerance by altering the gut microbiota. Nature 2014; 514(7521): 181-6.
34. Baer D J, Stote K S, Henderson T, et al. The metabolizable energy of dietary resistant maltodextrin is variable and alters fecal microbiota composition in adult men. J Nutr 2014; 144:1023-29.
35. Stack N, Mueller K, Schemann M and Haller D. Bacterial proteases in IBD and IBS. Gut 2012; 61(11): 1610-8.
36. Wang L, Christophersen C T, Sorich M J, et al. Increased abundance of Sutterella spp. And Ruminococcus torques in feces of children with autism spectrum disorder. Mol Autism 2013; 4(1):42.
37. Kaur N, Chen C C, Luther J, Kao J Y. Intestinal dysbiosis in inflammatory bowel disease. Gut Microbes 2011; 2(4):211-6.
38. Ducs F A, Sakar Y. Lepage P, et al. Replication of obesity and associated signaling pathways through transfer of microbiota from obese-prone rats. Diabetes 2014; 63(5): 1624-36.
39. Ryan K K, Tremaroli V. Clemmensen C, et al. FXR is a molecular target for the effects of vertical sleeve gastrectomy. Nature 2014; 509(7499): 183-8.
40. Ravussin Y, Koren O, Spor A, Et al. Responses of gut microbiota to diet composition and weight loss in lean and obese mice. Obesity 2012; 20(4):738-47.
41. Belzer C, Gerber G K, Roeselers G, et al. Dynamics of the microbiota in response to host infection. PLoS One 2014; 9(7):e95534.
42. Everard A, Belzer C, Geurts L. Cross-talk between Akkermansia muciniphilia and intestinal epithelium controls diet-induced obesity. Proc Natl Acad Sci USA 2013; 110(22):9066-71.
43. Stojanovic M R, Biagi E, Heilig HGHJ, et al. Global and deep molecular analysis of microbiota signatures in fecal samples from patients with irritable bowel syndrome. Gastroenterology 2011; 141(5): 1792-801.
44. Karlsson F H, Tremaroli V, Nookaew 1, et al. Gut metagenome in European women with normal, impaired and diabetic glucose control. Nature 2013; 498(7452):99-103.
45. Kang D-W, Park J G. Ilhan Z E, et al. Reduced incidence of Prevotella and other fermenters in intestinal microflora of autistic children. PLoS One 2013; 8(7):e68322.
46. Koeth R A, Wang Z, Levison B S, et al. Intestinal microbiota metabolism of L-carnitine, a nutrient in red meat, promotes atherosclerosis. Nat Med 2013; 19(5):576-85.
47. Salonen A, Lahti L, Salojärvi J, et al. Impact of diet and individual variation on intestinal microbiota composition and fermentation products in obese men. ISME J 2014; 8(11):2218-30.
48. Kim H J, Sung M-K. Kim J-S. Anti-inflammatory effects of glyceollins derived from soybean by elicitation with Aspergillus sojae. Inflamm Res 2011; 60:909-17.
49. Wonhwa L, Ku S-K, Lee Y-M, et al. Anti-septic effects of glyceollins in HMGB-1-induced inflammatory responses in vitro and in vivo. Food Chem Tox 2014; 63: 108.

What is claimed is:

1. A composition comprising:
isolated plant tissue having at least 0.01 mg glyceollin content per gram of plant tissue.

2. A composition comprising isolated soy pod tissue containing one or more glyceollins.

3. The composition of claim 2 wherein the combined total concentration of one or more glyceollins is at least 0.06 mg per gram of soy pod tissue.

4. The composition of claim 2 wherein the combined total concentration of one or more glyceollins is at least 0.1 mg per gram of soy pod tissue.

5. The composition of claim 2 wherein the combined total concentration of one or more glyceollins is at least 5 mg per gram of soy pod tissue.

6. The composition of any of claims 2 to 5, wherein the soy pod contains soluble and insoluble dietary fiber.

7. The composition of any of claims 1 to 6 formulated for oral delivery.

8. A food product comprising dietary fiber from soy pod tissue.

9. The food product of claim 8 further comprising one or more glyceollins.

10. The food product of claim 9 wherein the combined total amount of glyceollins is at least 10 mg.

11. A composition comprising a powder containing one or more glyceollins.

12. The composition of claim 11 wherein the powder is made from soy pod tissue.

13. The composition of any of claims 11 to 12 wherein the powder contains one or more glyceollins at a combined total concentration of at least ±0.01 mg glyceollins per gram of powder.

14. The composition of any of claims 11 to 12 wherein the powder contains one or more glyceollins at a combined total concentration of at least 0.06 mg glyceollins per gram of powder.

15. The composition of any of claims 11 to 12 wherein the powder contains one or more glyceollins at a combined total concentration of at least 1.0 mg glyceollins per gram of powder.

16. A method for treating a subject suffering from or susceptible to overweight or obesity, the method comprising a step of orally administering to the subject a composition or food product according to any of claims 1 to 15.

17. A method for treating a subject susceptible to or suffering from diabetes, the method comprising a step of orally administering to the subject a composition or food product according to any of claims 1 to 15.

18. A method for treating a subject susceptible to or suffering from Inflammatory Bowel Disease, the method comprising a step of orally administering to the subject a composition or food product according to any of claims 1 to 15.

19. A method for modifying the gastrointestinal microbiome of a subject wherein the gastrointestinal microbiome of the subject includes a first population of bacteria that process fat and protein, and a second population of bacteria that ferment carbohydrate and produce increases in small chain fatty acids, the method comprising the step of:
administering to the subject a composition comprising an effective amount of one or more glyceollins to shift the relative abundance of the first population of bacteria and the second population of bacteria in the gastrointestinal tract of the subject.

20. The method of claim 19 wherein the first population comprises Ruminococcaceae and the second population comprises Blautia hydrogenotrophica.

21. A method for modifying the gastrointestinal microbiome of a subject, the method comprising the step of:
administering to a subject whose microbiota taxa is determined to contain a level of Blautia below 5% abundance,
a composition comprising an effective amount of one or more glyceollins to increase Blautia levels to at least 20% abundance.

22. A method for treating gastrointestinal dysbiosis in a subject comprising the step of orally administering to the subject an effective amount of a composition comprising one or more glyceollins.

23. A method of manufacturing a powder comprising soy pod dietary fiber and one or more glyceollins, the method comprising the steps of:
obtaining soy pod tissue,
slicing the soy pod tissue,
adding one or more glyceollins to the soy pod tissue,
drying the soy pod tissue, and
pulverizing the soy pod tissue into a powder.
